(19) United States
(12) Patent Application Publication
(10) Pub. No.: US 2022/0135982 A1
(43) Pub. Date: May 5, 2022

(54) COMPOSITIONS FOR SUPPRESSING TRIM28 AND USES THEREOF

(71) Applicant: Memorial Sloan Kettering Cancer Center, New York, NY (US)

(72) Inventors: Chichao CHEN, New York, NY (US); Scott LOWE, New York, NY (US)

(21) Appl. No.: 17/428,322

(22) PCT Filed: Feb. 6, 2020
(86) PCT No.: PCT/US20/17049

§ 371 Date: Aug. 4, 2021

Related U.S. Application Data
(60) Provisional application No. 62/802,520, filed on Feb. 7, 2019.

(51) Int. Cl. C12N 15/113; A61P 35/02; C12N 9/10

(52) U.S. Cl. CPC C12N 15/1137; A61P 35/02; C12N 2320/30; C12N 2310/20; C12N 2320/12; C12N 9/104

(57) ABSTRACT

The present disclosure relates generally to compositions and methods for treating or ameliorating acute myeloid leukemia in a subject in need thereof. In particular, the present disclosure provides a method comprising administering to a subject a therapeutically effective amount of at least one agent that suppresses Trim28 activity.

### CROSS-REFERENCE TO RELATED APPLICATIONS

This application is a U.S. National Stage Application under 35 U.S.C. § 371 of International Patent Application No. PCT/US2020/017049, filed on Feb. 6, 2020, which claims the benefit of and priority to U.S. Provisional Patent Application No. 62/802,520, filed Feb. 7, 2019, the entire contents of each of which are incorporated herein by reference.

### GOVERNMENT SUPPORT

This invention was made with government support under grant number R01 CA190261, awarded by the National Institutes of Health/National Cancer Institute. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 28, 2020, is named 115872-0642_SL.txt and is 29,091 bytes in size.

### TECHNICAL FIELD

The present disclosure relates generally to compositions and methods for treating and/or ameliorating acute myeloid leukemia in a subject in need thereof. In particular, the present technology relates to administering a therapeutically effective amount of one or more compositions that inhibit Trim28 to a subject diagnosed with, or at risk for acute myeloid leukemia.

### BACKGROUND

The following description of the background of the present technology is provided simply as an aid in understanding the present technology and is not admitted to describe or constitute prior art to the present technology.

Acute myeloid leukemia (AML) has the worst 5-year-survival rate of all leukemias. Despite therapeutic improvement in recent decades, AML remains a clinically challenging disease.

### SUMMARY OF THE PRESENT TECHNOLOGY

In one aspect, the present disclosure provides a method for treating or preventing acute myeloid leukemia in a subject in need thereof, comprising administering to the subject a therapeutically effective amount of at least one Trim28-specific inhibitory nucleic acid that inhibits Trim28 activity in the subject. In some embodiments, the at least one Trim28-specific inhibitory nucleic acid is complementary to a Trim28 protein domain, such as the RBCC domain, HP1 protein binding domain (HP1BD), Plant Homeodomain (PHD) and Bromodomain. Additionally or alternatively, in some embodiments, the at least one Trim28-specific inhibitory nucleic acid is a sgRNA or shRNA comprising a nucleic acid sequence selected from the group consisting of: 5′ TTACAGTAGACTGTTCGCTCTC 3′ (SEQ ID NO: 1), 5′ TTCTGCACATCAGACACCTGGC 3′ (SEQ ID NO: 2), 5′ TTGAACTGTTTGAACATGCGGC 3′ (SEQ ID NO: 3), 5′ TTAACTTGTTGAATTGCTTGAA 3′ (SEQ ID NO: 4), 5′ TTCAATAACAATAAGGTTGTAG 3′ (SEQ ID NO: 5), 5′ TAGATCAACTTCTTAGAAAGCA 3′ (SEQ ID NO: 6), 5′ CTACAGGCCGAGTGCAAACA 3′ (SEQ ID NO: 7), 5′ GAGAGCGCCTGCGACCCGAG 3′ (SEQ ID NO: 8), 5′ CCAGCGGGTGAAGTACACCA 3′ (SEQ ID NO: 9), and 5′ CCCAGCCACCAGCTACTGTG 3′ (SEQ ID NO: 10).

Additionally or alternatively, in some embodiments, the subject displays elevated expression levels of Trim28 protein in leukemic cells prior to treatment, and/or has been diagnosed as having AML. Signs or symptoms of AML include, but are not limited to, leukemic cell proliferation, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, or loss of appetite.

Additionally or alternatively, in certain embodiments, the subject harbors one or more point mutations in NRAS, DNMT3A, FLT3, KIT, IDH1, IDH2, CEBPA and NPM1, and/or one or more gene fusions selected from the group consisting of CBFB-MYH11, DEK-NUP214, MLL-MLLT3, PML-RARA, RBM15-MKL1, RPN1-EVI1 and RUNX1-RUNX1T1. In some embodiments, the subject is human.

In any and all embodiments of the methods disclosed herein, the at least one Trim28-specific inhibitory nucleic acid is administered orally, topically, intranasally, systemically, intravenously, subcutaneously, intraperitoneally, intradermally, intraocularly, iontophoretically, transmucosally, or intramuscularly. The at least one Trim28-specific inhibitory nucleic acid may be administered daily for 6 weeks or more, or may be administered daily for 12 weeks or more.

Additionally or alternatively, in some embodiments, the method further comprises separately, sequentially or simultaneously administering one or more additional therapeutic agents to the subject. Examples of the one or more additional therapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and combinations thereof.

In one aspect, the present disclosure provides a method for monitoring the therapeutic efficacy of a Trim28-specific inhibitory nucleic acid in a subject diagnosed with AML comprising: (a) detecting H3K9 trimethylation levels in a test sample obtained from the subject after the subject has been administered the Trim28-specific inhibitory nucleic acid; and (b) determining that the Trim28-specific inhibitory nucleic acid is effective when the H3K9 trimethylation levels in the test sample are reduced compared to that observed in a control sample obtained from the subject prior to administration of the Trim28-specific inhibitory nucleic acid. In some embodiments, H3K9 trimethylation levels are detected via chromatin immunoprecipitation. In another aspect, the present disclosure provides a method for inhibiting leukemic cell proliferation in a subject in need thereof, comprising administering to the subject a therapeutically effective amount of at least one Trim28-specific inhibitory nucleic acid, wherein the subject suffers from a disease or condition characterized by elevated expression levels and/or increased activity of TRIM28. The Trim28-specific inhibitory nucleic acid may be an antisense oligonucleotide, a shRNA or a sgRNA.

Additionally or alternatively, in some embodiments of the methods disclosed herein, the Trim28-specific inhibitory nucleic acid is complementary to a Trim28 protein domain, such as the RBCC domain, HP1 protein binding domain (HP1BD), Plant Homeodomain (PHD) and Bromodomain. Additionally or alternatively, in some embodiments, the at least one Trim28-specific inhibitory nucleic acid is a sgRNA or shRNA comprising a nucleic acid sequence selected from the group consisting of: 5′ TTACAGTAGACTGTTCGCTCTC 3′ (SEQ ID NO: 1), 5′ TTCTGCACATCAGACACCTGGC 3′ (SEQ ID NO: 2), 5′ TTGAACTGTTTGAACATGCGGC 3′ (SEQ ID NO: 3), 5′ TTAACTTGTTGAATTGCTTGAA 3′ (SEQ ID NO: 4), 5′ TTCAATAACAATAAGGTTGTAG 3′ (SEQ ID NO: 5), 5′ TAGATCAACTTCTTAGAAAGCA 3′ (SEQ ID NO: 6), 5′ CTACAGGCCGAGTGCAAACA 3′ (SEQ ID NO: 7), 5′ GAGAGCGCCTGCGACCCGAG 3′ (SEQ ID NO: 8), 5′ CCAGCGGGTGAAGTACACCA 3′ (SEQ ID NO: 9), and 5′ CCCAGCCACCAGCTACTGTG 3′ (SEQ ID NO: 10).

### BRIEF DESCRIPTION OF THE DRAWINGS

FIGS. 1A-1C: Schematics and results of in vivo screening. FIG. 1A: The AML model is driven by MLL-AF9 and Nras(G12D). The library construct expresses 2 fluorescent proteins. GFP is constitutively expressed and dsRed expression is driven by a doxycycline inducible TRE promoter along with a mir30 cassette harboring the shRNA. Doxycycline was added to the diet after the AML disease was established. FIG. 1B: The candidate gene hits that scored with more than 3 shRNAs. FIG. 1C: The survival curve of TCGA patients categorized according to the expression level of Trim28.

FIG. 2: Schematic of Trim28 structure domains. RING—Really Interesting New Gene; zinc finger type domain; B1, B2—B-box type 1 and B-box type zinc finger type domains; CC—Coiled Coil; RBCC—RING domain followed by B-boxes and CC domain; TSS—TRIM Specific Sequence; HP1BD—HP1 protein binding domain; PHD—Plant Homeodomain; BROMO—Bromodomain, responsible for NuRD/SETDB1 recruitment and binding (P. Czerwinska, S. Mazurek, M. Wiznerowicz, J Biomed Sci 24, 63 (2017)).

FIG. 3: Essentiality landscapes of screen hits. Gray color in the bottom panel means requirement for cell proliferation. Each column represents a different cancer cell line. AML cell lines were marked by gray bars in the panel designated as ‘type.’

FIGS. 4A-4B: Genetic validation of Trim28 dependency. FIG. 4A: Mouse AML cells driven by different oncogenes are sensitive to Trim28 suppression with RNAi. Mouse embryonic fibroblast (MEF) cells (top right) are less sensitive than AMLs. The western blot in the middle shows the efficacy of Trim28 shRNA to knock down Trim28 protein in Nras(G12D)/MLL-AF9 cells. FIG. 4B: Mouse Nras(G12D)/MLL-AF9 AML cells were transduced with Cas9 protein first and infected with indicated sgRNAs against Trim28. Fitness of sgRNA infected AMLs is evaluated by tracking GFP % (sgRNA expressing) between day 4 and day 14. Percentages are normalized to day 4. The knock-out of Trim28 was demonstrated by western blot on the right.

FIGS. 5A-5H: In vivo validation of Trim28 dependency. FIG. 5A: Schematic of in vivo validation using inducible shRNA. The shRNA along with GFP is driven by a doxycycline inducible T3G promoter. AML cells were transduced with shRNA constructs and underwent neomycin selection to generate a pure population of infected cells. Cells were transplanted into sub-lethally irradiated mice, and doxycycline was added to the diet to turn on the shRNA after disease is established and detected (day 4) to mimic the Trim28 suppression in clinical situation. FIG. 5B: Luciferase imaging was done on day 4 (before doxycycline) and day 10 (after doxycycline) after transplant. FIG. 5C: Quantification of luciferase imaging shown in FIG. 5B. FIG. 5D: Kaplan-Meier curves of mice transplanted with AML cells harboring indicated shRNAs. FIG. 5E: Complete blood count (CBC) analysis of mice transplanted with AML cells harboring indicated shRNAs on day 10 after transplant. FIG. 5F: Flow cytometry analysis for GFP % on day 10 after transplant. FIG. 5G-5H: Bone marrow (BM) and spleen samples harvested from the moribund mice transplanted with AML cells harboring the indicated shRNA were analyzed for GFP %.

FIG. 6: Genetic validation in human AML lines. Competition assay strategy: Human AML cell lines were engineered to express Cas9 and GFP-linked sgRNA against a gene of interest. Cells were allowed to grow for 30 days. The fitness of different human cell lines harboring the indicated sgRNAs is reflected in the relative percentage of GFP+ cells in the population tracked from day 2 through day 30.

FIGS. 7A-7E: Genetic validation in human AMLs. FIG. 7A: Schematic showing the vector used to introduce shRNA into human cord blood derived leukemia. FIG. 7B: Western blot done with Molm13 showing the efficacy of the human TRIM28 shRNA. FIG. 7C: Western blot done with Thp1 cell lines showing the efficacy of the human TRIM28 shRNA. FIG. 7D-7E: The relative percentage of shRNA harboring cord blood leukemic cells on day 1 through day 9 after infection.

FIG. 8: Trim28 suppression differentiates AML cells. Flow cytometry analysis of Trim28 knock-down cells shows decreased c-Kit expression and increased Mac-1 expression, which suggests differentiation of AML cells. Data is shown for day 3, day 4, and day 5 after induction of shRNA against Trim28 or Renilla luciferase.

FIG. 9: Trim28 suppression differentiates AML cells. Flow cytometry analysis of Trim28 knock-out cells shows increased Mac-1 expression, which suggests differentiation of AMLs. Data is shown for day 4, day 6, and day 8 after infection of sgRNA against Trim28 or chromosome 8 (negative control).

FIGS. 10A-10C: GSEA analysis of RNA-seq data. FIG. 10A-10B: Gene set enrichment analysis (GSEA) showing enrichment of genes downregulated in stem cells in Trim28 KO MLL-AF9 leukemia cells. FIG. 10C: GSEA analysis showing negative enrichment of Myc target genes in Trim28 KO MLL-AF9 leukemia cells.

FIGS. 11A-11C: Domain screening using Trim28 sgRNAs. FIG. 11A: The domain structure of human and mouse Trim28. FIG. 11B: MLL-AF9 AML cells were infected with GFP-tethered sgRNAs, and the abundance of sgRNA harboring cells were tracked through day 14. FIG. 11C: Abundance fold change calculated from the data in FIG. 11B. Red arrows mark the most sensitive domains in the protein (J. Shi et al., Nat Biotechnol 33, 661 (2015)).

FIG. 12: Hypothesis of mechanism. Left: Model showing that Trim28 recruits Setdb1 to tri-methylate H3K9, suppressing the expression of genes that would lead to differentiation of AML cells. Right: inhibition of Setdb1 partially phenocopies Trim28 inhibition in the mouse AML model, reducing AML fitness.

FIG. 13A-13B: ChIP-seq and ChIP-q-PCR data. FIG. 13A: Three different Trim28 antibodies and one H3K9me3 antibody were tested for immunoprecipitation of Trim28 cross-linked DNA in MEF cells. FIG. 13B: ChIP-seq data using H3K9me3 antibody was performed in MLL-AF9 mouse AML cells (RN2). The signal was compared to publicly available ChIP-seq data from mouse embryonic stem cells (mESCs).

FIG. 14: Cellular Roles of TRIM28.

### DETAILED DESCRIPTION

It is to be appreciated that certain aspects, modes, embodiments, variations and features of the present methods are described below in various levels of detail in order to provide a substantial understanding of the present technology.

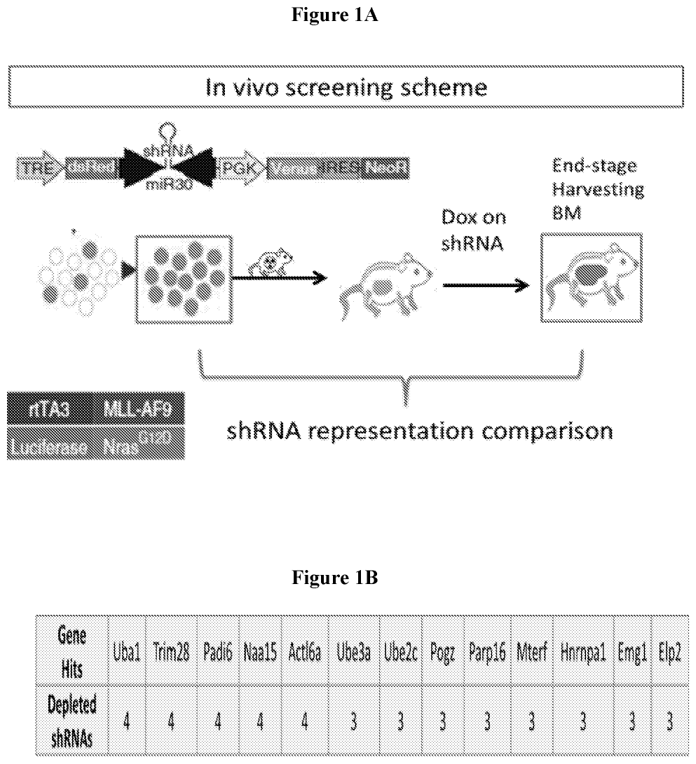

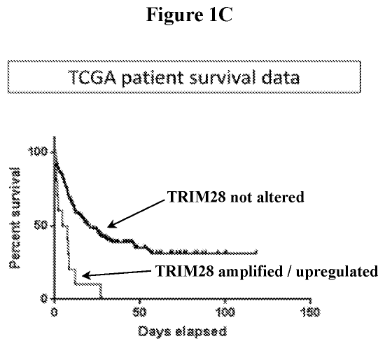

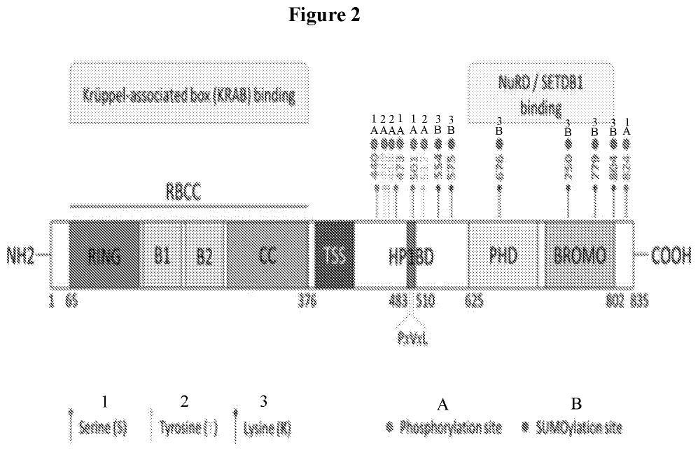

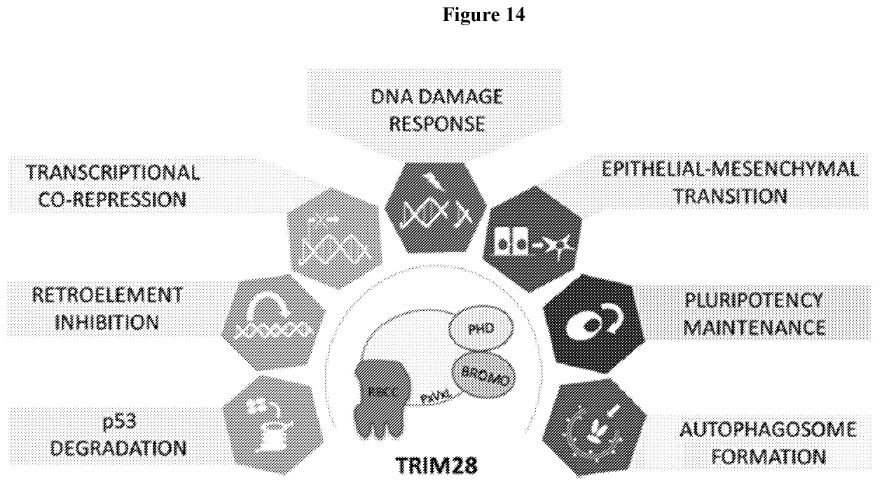

The present disclosure provides epigenetic regulators as potential therapeutic targets for AML. Trim28 was chosen as a follow up target after comparing hits to publicly available CRISPR dependency screening done in human cancer cell lines and integrating patient prognosis data (FIG. 1C) from TCGA (N. Cancer Genome Atlas Research et al., Nat Genet 45, 1113 (2013)) and cBioportal (E. Cerami et al., Cancer Discov 2, 401 (2012); J. Gao et al., Sci Signal 6, p 11 (2013)). Trim28-Transcription intermediary factor 1-beta (FIG. 2) was identified as a required gene in AML maintenance. TRIM28 is involved in the regulation of gene expression through heterochromatin formation, mediation of DNA damage response, inhibition of p53 activity through intrinsic E3 ubiquitin ligase activity, regulation of epithelial to mesenchymal transition (EMT) and maintenance of stem cell pluripotency as well as regulation of autophagy and safeguarding the genome stability through inhibition of retrotransposition (FIG. 14). Though its domain structure has been known for some time, the role of TRIM28 in cancer cells has been unclear for more than a decade, despite attempts to understand it. While some studies suggest that higher TRIM28 levels are linked to a poor prognosis in certain cancers (F. Li, Z. Wang, G. Lu, Oncol Rep 39, 1860 (2018); L. Liu et al., Mol Med Rep 17, 835 (2018)), the opposite has also been reported (L. Chen et al., J Biol Chem 287, 40106 (2012)). Furthermore, liver-specific ablation of Trim28 in mice increases male-predominant hepatic adenoma, suggesting that TRIM28 protects liver cells from tumorigenic conversion (K. Bojkowska et al., Hepatology 56, 1279 (2012)).

As demonstrated herein, Trim28 suppression with shRNAs or sgRNA led to robust terminal myeloid differentiation and resulted in antileukemic effects in vitro and in vivo. Without wishing to be bound by theory, it is believed that the consequences of Trim28 suppression may be due, at least in part, to its role in recruiting Setdb1 to tri-methylate histone tails at multiple genetic loci in AML. These results demonstrate that Trim28 is an essential gene for AML to sustain its undifferentiated status, thereby making Trim28 a potential therapeutic target in AML.

In the hematopoietic system, genetic loss of Trim28 in the adult mouse leads to defective immature erythropoiesis, possibly due to down-regulation of multiple erythroid transcription factors and heme biosynthesis enzymes (T. Hosoya, M. Clifford, R. Losson, O. Tanabe, J. D. Engel, Blood 122, 3798 (2013)). Analyses of hematopoietic progenitor population in this Trim28flox/flox:TgMxlCre (TMC) congenic mice also demonstrated reduction in the number of lymphocytes, which is consistent with another study using a mouse model with lymphocyte cell-specific ablation of Trim28. Surprisingly, development of myeloid lineage (HSC/MPP or GMP progenitors, or Mac1+ cells) appeared to be quite normal in both the bone marrow and peripheral blood. These mouse studies, together with the present findings in a genome wide CRISPR screen in human cells confirmed that Trim28 is not a universally required gene, thus further validating its potential as a therapeutic target.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intraperitoneally, or subcutaneously), rectally, intrathecally, intratumorally or topically. Administration includes self-administration and the administration by another.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complementary sequence can also be an RNA sequence complementary to the DNA sequence or its complementary sequence, and can also be a cDNA.

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, the term “effective amount” refers to a quantity sufficient to achieve a desired therapeutic and/or prophylactic effect, e.g., an amount which results in the prevention of, or a decrease in a disease or condition described herein or one or more signs or symptoms associated with a disease or condition described herein. In the context of therapeutic or prophylactic applications, the amount of a composition administered to the subject will vary depending on the composition, the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compositions may be administered to a subject having one or more signs or symptoms of a disease or condition described herein. As used herein, a “therapeutically effective amount” of a composition refers to composition levels in which the physiological effects of a disease or condition are ameliorated or eliminated. A therapeutically effective amount can be given in one or more administrations.

As used herein, “expression” includes one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “gene” means a segment of DNA that contains all the information for the regulated biosynthesis of an RNA product, including promoters, exons, introns, and other untranslated regions that control expression.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same nucleobase or amino acid, then the molecules are homologous at that position. A degree of homology between sequences is a function of the number of matching or homologous positions shared by the sequences. A polynucleotide or polynucleotide region (or a polypeptide or polypeptide region) has a certain percentage (for example, at least 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art. In some embodiments, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the National Center for Biotechnology Information. Biologically equivalent polynucleotides are those having the specified percent homology and encoding a polypeptide having the same or similar biological activity. Two sequences are deemed “unrelated” or “non-homologous” if they share less than 40% identity, or less than 25% identity, with each other.

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tm) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” can be an individual organism, a vertebrate, a mammal, or a human. In some embodiments, the individual, patient or subject is a human.

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. One or more bases of the oligonucleotide may also be modified to include a phosphorothioate bond (e.g., one of the two oxygen atoms in the phosphate backbone which is not involved in the internucleotide bridge, is replaced by a sulfur atom) to increase resistance to nuclease degradation. The exact size of the oligonucleotide will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration. Pharmaceutically-acceptable carriers and their formulations are known to one skilled in the art and are described, for example, in Remington's Pharmaceutical Sciences (20^{th} edition, ed. A. Gennaro, 2000, Lippincott, Williams & Wilkins, Philadelphia, Pa.).

As used herein, the term “polynucleotide” or “nucleic acid” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons.

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “sample” means biological sample material derived from living cells of a subject. Biological samples may include tissues, cells, protein or membrane extracts of cells, and biological fluids (e.g., ascites fluid or cerebrospinal fluid (CSF)) isolated from a subject, as well as tissues, cells and fluids (blood, plasma, saliva, urine, serum etc.) present within a subject.

As used herein, the term “separate” therapeutic use refers to an administration of at least two active ingredients at the same time or at substantially the same time by different routes.

As used herein, the term “sequential” therapeutic use refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. There is no simultaneous treatment in this case.

As used herein, the term “simultaneous” therapeutic use refers to the administration of at least two active ingredients by the same route and at the same time or at substantially the same time.

The term “specific” as used herein in reference to an oligonucleotide means that the nucleotide sequence of the oligonucleotide has at least 12 bases of sequence identity with a portion of a target nucleic acid when the oligonucleotide and the target nucleic acid are aligned. An oligonucleotide that is specific for a target nucleic acid is one that, under the stringent hybridization or washing conditions, is capable of hybridizing to the target nucleic acid of interest and not substantially hybridizing to nucleic acids which are not of interest. Higher levels of sequence identity are desirable and include at least 75%, at least 80%, at least 85%, at least 90%, at least 95% or at least 98% sequence identity.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be modulated (e.g., inhibited or downregulated).

As used herein, the term “therapeutic agent” is intended to mean a compound that, when present in an effective amount, produces a desired therapeutic effect on a subject in need thereof (e.g., ameliorating or treating AML).

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

### Trim28-Specific Inhibitory Nucleic Acids of the Present Technology

In one aspect, the present disclosure provides inhibitory nucleic acids (e.g., sgRNAs, antisense RNAs or shRNAs) that inhibit Trim28 activity. The mammalian nucleic acid and amino acid sequences of Trim28 are known in the art. For example, the mRNA and amino acid sequences of human Trim28, represented as SEQ ID NO: 11 and SEQ ID NO: 12, respectively, are provided below:

| Homo sapiens tripartite motif containing 28 (TRIM28), mRNA | |
|---|---|
| (SEQ ID NO: 11) | |
| 1 | agtgacgcag aggctggaga cgactctacg gcggcgaaga gacgcgggtt gaggaagagg |
| 61 | gacggattgc ccatgcgctt gggcgcacag cggcccgctt ctgtgtggtc tggaggtgga |
| 121 | gctgagaggg gaatcacact ctataaaggt tcgcataccc cactggcgga ttcaattgcg |
| 181 | gcagtgacgt cacagaggcc ccgccccgcc cccacaagag ccccaccgac gtggggttgg |
| 241 | cggtggtgga aggactagga gttggcgcgt gcgtactggc ggcctctccc gcaccgaccg |
| 301 | gcctgggccc cgcccccggg cgtgaggcgc ccaatgcgcg tgcgcggcgg cgtcggcgcc |
| 361 | agttatttct gtcccgcccc ccggcctcgg ctctttctgc gagcgggcgc gcgggcgagc |
| 421 | ggttgtgctt gtgcttgtgg cgcgtggtgc gggtttcggc ggcggctgag gaagaagcgc |
| 481 | gggcggcgcc ttcgggaggc gagcaggcag cagttggccg tgccgtagca gcgtcccgcg |
| 541 | cgcggcgggc agcggcccag gaggcgcgtg gcggcgctcg gcctcgcggc ggcggcggcg |
| 601 | gcagcggccc agcagttggc ggcgagcgcg tctgcgcctg cgcggcgggc cccgcgcccc |
| 661 | tcctcccccc ctgggcgccc ccggcggcgt gtgaatggcg gcctccgcgg cggcagcctc |
| 721 | ggcagcagcg gcctcggccg cctctggcag cccgggcccg ggcgagggct ccgctggcgg |
| 781 | cgaaaagcgc tccaccgccc cttcggccgc agcctcggcc tctgcctcag ccgcggcgtc |
| 841 | gtcgcccgcg gggggcggcg ccgaggcgct ggagctgctg gagcactgcg gcgtgtgcag |
| 901 | agagcgcctg cgacccgaga gggagccccg cctgctgccc tgtttgcact cggcctgtag |
| 961 | tgcctgctta gggcccgcgg cccccgccgc cgccaacagc tcgggggacg gcggggcggc |
| 1021 | gggcgacggc accgtggtgg actgtcccgt gtgcaagcaa cagtgcttct ccaaagacat |
| 1081 | cgtggagaat tatttcatgc gtgatagtgg cagcaaggct gccaccgacg cccaggatgc |
| 1141 | gaaccagtgc tgcactagct gtgaggataa tgccccagcc accagctact gtgtggagtg |
| 1201 | ctcggagcct ctgtgtgaga cctgtgtaga ggcgcaccag cgggtgaagt acaccaagga |
| 1261 | ccatactgtg cgctctactg ggccagccaa gtctcgggat ggtgaacgta ctgtctattg |
| 1321 | caacgtacac aagcatgaac cccttgtgct gttttgtgag agctgtgata ctctcacctg |
| 1381 | ccgagactgc cagctcaatg cccacaagga ccaccagtac cagttcttag aggatgcagt |
| 1441 | gaggaaccag cgcaagctcc tggcctcact ggtgaagcgc cttggggaca aacatgcaac |
| 1501 | attgcagaag agcaccaagg aggttcgcag ctcaatccgc caggtgtctg acgtacagaa |
| 1561 | gcgtgtgcaa gtggatgtca agatggccat cctgcagatc atgaaggagc tgaataagcg |
| 1621 | gggccgtgtg ctggtcaatg atgcccagaa ggtgactgag gggcagcagg agcgcctgga |
| 1681 | gcggcagcac tggaccatga ccaagatcca gaagcaccag gagcacattc tgcgctttgc |
| 1741 | ctcttgggct ctggagagtg acaacaacac agcccttttg ctttctaaga agttgatcta |
| 1801 | cttccagctg caccgggccc tcaagatgat tgtggatccc gtggagccac atggcgagat |
| 1861 | gaagtttcag tgggacctca atgcctggac caagagtgcc gaggcctttg gcaagattgt |
| 1921 | ggcagagcgt cctggcacta actcaacagg ccctgcaccc atggcccctc caagagcccc |
| 1981 | agggcccctg agcaagcagg gctctggcag cagccagccc atggaggtgc aggaaggcta |
| 2041 | tggctttggg tcaggagatg atccctactc aagtgcagag ccccatgtgt caggtgtgaa |
| 2101 | acggtcccgc tcaggtgagg gcgaggtgag cggccttatg cgcaaggtgc cacgagtgag |
| 2161 | ccttgaacgc ctggacctgg acctcacagc tgacagccag ccacccgtct tcaaggtctt |
| 2221 | cccaggcagt accactgagg actacaacct tattgttatt gaacgtggcg ctgccgctgc |
| 2281 | agctaccggc cagccaggga ctgcgcctgc aggaacccct ggtgccccac ccctggctgg |
| 2341 | catggccatt gtcaaggagg aggagacgga ggctgccatt ggagcccctc ctactgccac |
| 2401 | tgagggccct gagaccaaac ctgtgcttat ggctcttgcg gagggtcctg gtgctgaggg |
| 2461 | tccccgcctg gcctcaccta gtggcagcac cagctcaggg ctggaggtgg tggctcctga |
| 2521 | gggtacctca gccccaggtg gtggcccggg aaccctggat gacagtgcca ccatttgccg |
| 2581 | tgtctgccag aagccaggcg atctggttat gtgcaaccag tgtgagtttt gtttccacct |
| 2641 | ggactgtcac ctgccggccc tgcaggatgt accaggggag gagtggagct gctcactctg |
| 2701 | ccatgtgctc cctgacctga aggaggagga tggcagcctc agcctggatg gtgcagacag |
| 2761 | cactggcgtg gtggccaagc tctcaccagc caaccagcgg aaatgtgagc gtgtactgct |
| 2821 | ggccctattc tgtcacgaac cctgccgccc cctgcatcag ctggctaccg actccacctt |
| 2881 | ctccctggac cagcccggtg gcaccctgga tctgaccctg atccgtgccc gcctccagga |
| 2941 | gaagttgtca cctccctaca gctccccaca ggagtttgcc caggatgtgg gccgcatgtt |
| 3001 | caagcaattc aacaagttaa ctgaggacaa ggcagacgtg cagtccatca tcggcctgca |
| 3061 | gcgcttcttc gagacgcgca tgaacgaggc cttcggtgac accaagttct ctgctgtgct |
| 3121 | ggtggagccc ccgccgatga gcctgcctgg tgctggcctg agttcccagg agctgtctgg |
| 3181 | tggccctggt gatggcccct gaggctggag cccccatggc cagcccagcc tggctctgtt |
| 3241 | ctctgtcctg tcaccccatc cccactcccc tggtggcctg actcccactc cctggtggcc |
| 3301 | ccatccccca gttcctcacg atatggtttt tacttctgtg gatttaataa aaacttcacc |
| 3361 | agtt |
| Homo sapiens tripartite motif containing 28 (TRIM28), protein | |
| (SEQ ID NO: 12) | |
| 1 | MAASAAAASA AAASAASGSP GPGEGSAGGE KRSTAPSAAA SASASAAASS PAGGGAEALE |
| 61 | LLEHCGVCRE RLRPEREPRL LPCLHSACSA CLGPAAPAAA NSSGDGGAAG DGTVVDCPVC |
| 121 | KQQCFSKDIV ENYFMRDSGS KAATDAQDAN QCCTSCEDNA PATSYCVECS EPLCETCVEA |
| 181 | HQRVKYTKDH TVRSTGPAKS RDGERTVYCN VHKHEPLVLF CESCDTLTCR DCQLNAHKDH |
| 241 | QYQFLEDAVR NQRKLLASLV KRLGDKHATL QKSTKEVRSS IRQVSDVQKR VQVDVKMAIL |
| 301 | QIMKELNKRG RVLVNDAQKV TEGQQERLER QHWTMTKIQK HQEHILRFAS WALESDNNTA |
| 361 | LLLSKKLIYF QLHRALKMIV DPVEPHGEMK FQWDLNAWTK SAEAFGKIVA ERPGTNSTGP |
| 421 | APMAPPRAPG PLSKQGSGSS QPMEVQEGYG FGSGDDPYSS AEPHVSGVKR SRSGEGEVSG |
| 481 | LMRKVPRVSL ERLDLDLTAD SQPPVFKVFP GSTTEDYNLI VIERGAAAAA TGQPGTAPAG |
| 541 | TPGAPPLAGM AIVKEEETEA AIGAPPTATE GPETKPVLMA LAEGPGAEGP RLASPSGSTS |
| 601 | SGLEVVAPEG TSAPGGGPGT LDDS [symbol] [symbol] [symbol] [symbol] [symbol] [symbol] [symbol] |
| 661 | [symbol] [symbol] [symbol] PDLKEEDG SLSLDGADST GVVAKLSPAN QRKCERVLLA LFCHEPCRPL |
| 721 | HQLATDSTFS LDQPGGTLDL TLIRARLQEK LSPPYSSPQE FAQDVGRMFK QFNKLTEDKA |
| 781 | DVQSIIGLQR FFETRMNEAF GDTKFSAVLV EPPPMSLPGA GLSSQELSGG PGDGP |

*RBCC domain is underlined; HP1 domain is bold; PHD domain is bold and italicized; Bromodomain is italicized

For example, the mRNA and amino acid sequences of mouse Trim28, represented as SEQ ID NO: 13 and SEQ ID NO: 14, respectively, are provided below:

| Mus musculus tripartite motif-containing 28 (Trim28), mRNA | |
|---|---|
| (SEQ ID NO: 13) | |
| 1 | ggacctgcgc gctcaagcac gcgggttgcc agcctgggtg caccgtgcgg tgggcatggg |
| 61 | agacgtcaag cacgcactct gcgggtgaat tcctcaccag cagcattgct actctggatc |
| 121 | tagaggggcg gcagcgagtg gaagggaggg ccctggcgcg tgcgtaatct gcgccccgcc |
| 181 | cccaggctgc ggtgcccaac gcgcgtgcgc ggcgtcgtcg gcgccagata tttctgtccc |
| 241 | gcctccaggc cgcggctctt tctgcgagcg gcgcgcggtc aggcggttgt tcgcaagtct |
| 301 | tgcggcgcga gtgtctcaga agcgagggag gaggcggcgc tggtggcggc gccgtcgtct |
| 361 | gtacagaggc ccgcagccag cggcttggcc gcatctcgac agcgcctggg gcgcggcggg |
| 421 | cgtcggccca ggagacgcgt ggcggcgctc ggcctcgcgg catcggcggc tgcctggccg |
| 481 | ttggcggcga gcgcacttgc gcctgcgcag cgggctccgt gcccctcctc ccctgggcgg |
| 541 | cccccccacc cccccggcgg cgtgtgaatg gcggcctcgg cggcagcgac tgcagcggcc |
| 601 | tcggccgcga cggccgcctc ggcggcctct ggtagcccag ggtcgggcga gggctcggcg |
| 661 | ggcggtgaga agcgtccggc tgcttcctca gccgcggcgg cctctgcagc cgcgtcgtcc |
| 721 | cctgcggggg gcggtggcga ggcgcaggag cttctggagc actgcggcgt gtgtcgcgag |
| 781 | cgcctgcggc ccgagcggga tcctcggctg ctgccctgtc tacattcggc ctgcagtgcc |
| 841 | tgcctgggcc ccgctacacc cgccgcagcg aataattcgg gggatggcgg ctcggcgggc |
| 901 | gacggcgcta tggtggattg tccagtgtgc aaacagcagt gctactccaa agacatcgtg |
| 961 | gagaattatt ttatgcgtga tagtggcagt aaggcctctt ctgattccca ggatgctaac |
| 1021 | cagtgctgca ctagctgtga agataatgcc ccagccacta gctattgtgt ggagtgctct |
| 1081 | gaaccacttt gtgagacctg tgtggaggct caccagcggg tgaaatacac caaggaccac |
| 1141 | actgtgcgct ccacaggacc tgctaagact cgagatggag agcgaacagt ctactgtaat |
| 1201 | gtgcacaagc atgagcccct cgtgctgttc tgtgagagct gtgacacact cacctgccgc |
| 1261 | gactgccagc tcaacgctca caaggaccat cagtaccagt ttttggaaga tgcagtgagg |
| 1321 | aaccaacgta aactcttggc ttcactggtg aaacgtcttg gggacaaaca tgccacactt |
| 1381 | cagaaaaaca ccaaggaggt tcgaagctcg atccgccagg tgtctgatgt gcagaagcga |
| 1441 | gtgcaggttg atgtcaagat ggccattctg cagatcatga aggagctgaa taagcggggt |
| 1501 | cgagttctgg tcaatgatgc ccagaaggtg accgagggtc agcaggaacg tctggagcgc |
| 1561 | cagcactgga ccatgaccaa aattcagaag caccaggaac acattttgcg ttttgcctct |
| 1621 | tgggctctgg agagtgataa caatacagct ctcttgctct ctaagaagct gatctatttc |
| 1681 | cagctgcatc gggccctcaa aatgattgtg gatcctgtgg agcctcatgg tgagatgaag |
| 1741 | tttcagtggg atctcaatgc ctggaccaag agtgctgaag cctttggcaa gattgtggct |
| 1801 | gagcgtcctg gtacgaactc cacaggtcct gggcccatgg ctcctccaag agccccaggc |
| 1861 | cctctaagca agcaaggttc tggcagtagc cagcccatgg aagtacaaga gggatatggc |
| 1921 | tttgggtcag atgatcccta ttcaagtgca gagccgcatg tatcaggcat gaagcggtcc |
| 1981 | cgctctggtg agggagaggt aagtggcctc ttaaggaagg tgccacgtgt gagccttgaa |
| 2041 | cgcctggatc tggacctcac ctctgacagc cagccaccag tcttcaaggt ctttcctgga |
| 2101 | agcactactg aggactacaa tctgattgtt attgagcgtg gtgctgctgc agcagctgct |
| 2161 | ggtcaggctg ggactgtgcc accaggagcc cctggtgccc caccccttcc tggcatggcc |
| 2221 | attgtcaagg aagaagagac agaagctgct attggagctc ccccggctgc ccccgagggt |
| 2281 | cctgaaacca agcctgtgtt gatgcctctg actgaaggtc ctggtgccga gggacctcgt |
| 2341 | ctagcttcac ctagtggcag taccagctca ggcttggagg tggtggctcc tgaggttacc |
| 2401 | tcagccccag taagtgggcc aggtatcctg gatgacagtg ccactatctg ccgtgtctgc |
| 2461 | cagaaaccag gtgacctggt catgtgtaac cagtgcgaat tttgcttcca cctggattgc |
| 2521 | cacctccctg ccctgcagga tgttccaggg gaggaatgga gttgctcact ctgccacgtg |
| 2581 | ctccctgacc taaaggagga agatggaagc ctcagcctgg atggagcaga tagcactggt |
| 2641 | gtggtagcta aactctcacc agccaaccag cggaaatgtg agcgtgttct cctggccctg |
| 2701 | ttctgccatg aaccatgccg tcccttgcat cagctggcta ccgactctac attctccatg |
| 2761 | gagcagcctg gtggtaccct agacctgacc ttgattcgtg ctcgcctcca agagaagctg |
| 2821 | tcacctcctt atagctcccc ccaggagttt gctcaagatg tgggccgcat gttcaaacag |
| 2881 | ttcaacaagc tgactgagga caaggcagat gttcagtcca tcatcggctt gcagcgcttc |
| 2941 | tttgagacac gcatgaatga tgcctttggt gacaccaagt tttctgctgt gctggtagaa |
| 3001 | ccaccaccat tgaaccttcc cagtgctggc ctaagttctc aggagctctc tggccctggt |
| 3061 | gatggcccct gaagctgggg ctcttgtggt cagcccagtc cagctctggt ctctgtattt |
| 3121 | tcaccccata ccctgtcctt tggtggcctg actcctgttc ttgctggccc catcgtcccc |
| 3181 | tcagtccctc ttcacaaaat ggtttttact tctgtggatt taataaaaac ttcactgagt |
| 3241 | caaaaaaaaa aaaaa |
| Mus musculus tripartite motif-containing 28 (Trim28), protein | |
| (SEQ ID NO: 14) | |
| 1 | MAASAAATAA ASAATAASAA SGSPGSGEGS AGGEKRPAAS SAAAASAAAS SPAGGGGEAQ |
| 61 | ELLEHCGVCR ERLRPERDPR LLPCLHSACS ACLGPATPAA ANNSGDGGSA GDGAMVDCPV |
| 121 | CKQQCYSKDI VENYFMRDSG SKASSDSQDA NQCCTSCEDN APATSYCVEC SEPLCETCVE |
| 181 | AHQRVKYTKD HTVRSTGPAK TRDGERTVYC NVHKHEPLVL FCESCDTLTC RDCQLNAHKD |
| 241 | HQYQFLEDAV RNQRKLLASL VKRLGDKHAT LQKNTKEVRS SIRQVSDVQK RVQVDVKMAI |
| 301 | LQIMKELNKR GRVLVNDAQK VTEGQQERLE RQHWTMTKIQ KHQEHILRFA SWALESDNNT |
| 361 | ALLLSKKLIY FQLHRALKMI VDPVEPHGEM KFQWDLNAWT KSAEAFGKIV AERPGTNSTG |
| 421 | PGPMAPPRAP GPLSKQGSGS SQPMEVQEGY GFGSDDPYSS AEPHVSGMKR SRSGEGEVSG |
| 481 | LLRKVPRVSL ERLDLDLTSD SQPPVFKVFP GSTTEDYNLI VIERGAAAAA AGQAGTVPPG |
| 541 | APGAPPLPGM AIVKEEETEA AIGAPPAAPE GPETKPVLMP LTEGPGAEGP RLASPSGSTS |
| 601 | SGLEVVAPEV TSAPVSGPGI LDDS [symbol] [symbol] [symbol] [symbol] [symbol] [symbol] [symbol] |
| 661 | [symbol] [symbol] [symbol] PDLKEEDG SLSLDGADST GVVAKLSPAN QRKCERVLLA LFCHEPCRPL |
| 721 | HQLATDSTFS MEQPGGTLDL TLIRARLQEK LSPPYSSPQE FAQDVGRMFK QFNKLTEDKA |
| 781 | DVQSIIGLQR FFETRMNDAF GDTKFSAVLV EPPPLNLPSA GLSSQELSGP GDGP |

*RBCC domain is underlined; HP1 domain is bold; PHD domain is bold and italicized; Bromodomain is italicized

The inhibitory nucleic acids of the present technology may comprise a nucleic acid molecule which is complementary to a portion of a Trim28 protein domain (see e.g., SEQ ID NO: 12 or 14), such as the RBCC domain, HP protein binding domain (HP1BD), Plant Homeodomain (PHD), or Bromodomain. In some embodiments, the inhibitory nucleic acids of the present technology target at least one exon and/or intron of Trim28.

The present disclosure also provides an antisense nucleic acid comprising a nucleic acid sequence that is complementary to and specifically hybridizes with a portion of a Trim28 mRNA (e.g., SEQ ID NO: 11 or 13). The antisense nucleic acid may be antisense RNA, or antisense DNA. Antisense nucleic acids based on the known nucleic acid sequences of Trim28 can be readily designed and engineered using methods known in the art.

Antisense nucleic acids are molecules which are complementary to a sense nucleic acid strand, e.g., complementary to the coding strand of a double-stranded DNA molecule (or cDNA) or complementary to an mRNA sequence (e.g., SEQ ID NO: 11 or 13). Accordingly, an antisense nucleic acid can form hydrogen bonds with a sense nucleic acid. The antisense nucleic acid can be complementary to an entire coding strand of TRIM28 or to a portion thereof, e.g., all or part of the protein coding region (or open reading frame) (e.g., SEQ ID NO: 11 or 13). In some embodiments, the antisense nucleic acid is an oligonucleotide which is complementary to only a portion of the coding region of TRIM28 mRNA (e.g., SEQ ID NO: 11 or 13). In certain embodiments, an antisense nucleic acid molecule can be complementary to a noncoding region of the TRIM28 coding strand (e.g., SEQ ID NO: 11 or 13). In some embodiments, the noncoding region refers to the 5′ and 3′ untranslated regions that flank the coding region and are not translated into amino acids. For example, the antisense oligonucleotide can be complementary to the region surrounding the translation start site of TRIM28. An antisense oligonucleotide can be, for example, about 5, 10, 15, 20, 25, 30, 35, 40, 45 or 50 nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or modified nucleotides designed to increase the biological stability of the molecules or to increase the physical stability of the duplex formed between the antisense and sense nucleic acids, e.g., phosphorothioate derivatives and acridine substituted nucleotides. Examples of modified nucleotides which can be used to generate the antisense nucleic acid include 5-fluorouracil, 5-bromouracil, 5-chlorouracil, 5-hodouracil, hypoxanthine, xanthine, 4-acetylcytosine, 5-(carboxyhydroxylmethyl)uracil, 5-carboxymethylaminomethyl-2-thouridine, 5-carboxymethylaminometh-yluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-metnylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopenten-yladenine, uracil-5-oxyacetic acid (v), wybutosine, pseudouracil, queosine, 2-thiocytosine, 5-methyl-2-thiouracil, 2-thlouracil, 4-thiouracil, 5-methyluracil, uracil-5-oxyacetic acid methylester, uracil-5-cxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

The antisense nucleic acid molecules may be administered to a subject or generated in situ such that they hybridize with or bind to cellular mRNA and/or genomic DNA encoding the protein of interest to thereby inhibit expression of the protein, e.g., by inhibiting transcription and/or translation. The hybridization can occur via Watson-Crick base pairing to form a stable duplex, or in the case of an antisense nucleic acid molecule which binds to DNA duplexes, through specific interactions in the major groove of the double helix.

In some embodiments, the antisense nucleic acid molecules are modified such that they specifically bind to receptors or antigens expressed on a selected cell surface, e.g., by linking the antisense nucleic acid molecules to peptides or antibodies which bind to cell surface receptors or antigens. In some embodiments, the antisense nucleic acid molecule is an alpha-anomeric nucleic acid molecule. An alpha-anomeric nucleic acid molecule forms specific double-stranded hybrids with complementary RNA in which, contrary to the usual β-units, the strands run parallel to each other (Gaultier et al., Nucleic Acids. Res. 15:6625-6641(1987)). The antisense nucleic acid molecule can also comprise a 2′-O-methylribonucleotide (Inoue et al., Nucleic Acids Res. 15:6131-6148 (1987)) or a chimeric RNA-DNA analogue (Inoue et al., FEBS Lett. 215:327-330 (1987)).

The present disclosure also provides a short hairpin RNA (shRNA) or small interfering RNA (siRNA) comprising a nucleic acid sequence that is complementary to and specifically hybridizes with a portion of a TRIM28 mRNA (e.g., SEQ ID NO: 11 or 13), thereby reducing or inhibiting gene expression. In some embodiments, the shRNA or siRNA is about 18, 19, 20, 21, 22, 23, 24, 25, 26, 27, 28 or 29 base pairs in length. Double-stranded RNA (dsRNA) can induce sequence-specific post-transcriptional gene silencing (e.g., RNA interference (RNAi)) in many organisms such as C. elegans, Drosophila, plants, mammals, oocytes and early embryos. RNAi is a process that interferes with or significantly reduces the number of protein copies made by an mRNA. For example, a double-stranded siRNA or shRNA molecule is engineered to complement and hybridize to a mRNA of a target gene. Following intracellular delivery, the siRNA or shRNA molecule associates with an RNA-induced silencing complex (RISC), which then binds and degrades a complementary target mRNA, such as TRIM28 mRNA (e.g., SEQ ID NO: 11 or 13).

The present disclosure also provides a synthetic guide RNA (sgRNA) comprising a nucleic acid sequence that is complementary to and specifically hybridizes with a portion of a TRIM28 nucleic acid sequence (e.g., SEQ ID NO: 11 or 13). Guide RNAs for use in CRISPR-Cas systems are typically generated as a single guide RNA comprising a crRNA segment and a tracrRNA segment. The crRNA segment and a tracrRNA segment can also be generated as separate RNA molecules. The crRNA segment comprises the targeting sequence that binds to a portion of a TRIM28 nucleic acid sequence, and a stem portion that hybridizes to a tracrRNA. The tracrRNA segment comprises a nucleotide sequence that is partially or completely complementary to the stem sequence of the crRNA and a nucleotide sequence that binds to the CRISPR enzyme. In some embodiments, the crRNA segment and the tracrRNA segment are provided as a single guide RNA. In some embodiments, the crRNA segment and the tracrRNA segment are provided as separate RNAs. The combination of the CRISPR enzyme with the crRNA and tracrRNA make up a functional CRISPR-Cas system. Exemplary CRISPR-Cas systems for targeting nucleic acids, are described, for example, in WO2015/089465.

In some embodiments, a synthetic guide RNA is a single RNA represented as comprising the following elements: 5′-X1-X2-Y-Z-3′

where X1 and X2 represent the crRNA segment, where X1 is the targeting sequence that binds to a portion of a TRIM28 nucleic acid sequence, X2 is a stem sequence that hybridizes to a tracrRNA, Z represents a tracrRNA segment comprising a nucleotide sequence that is partially or completely complementary to X2, and Y represents a linker sequence. In some embodiments, the linker sequence comprises two or more nucleotides and links the crRNA and tracrRNA segments. In some embodiments, the linker sequence comprises 2, 3, 4, 5, 6, 7, 8, 9, 10 or more nucleotides. In some embodiments, the linker is the loop of the hairpin structure formed when the stem sequence hybridized with the tracrRNA.

In some embodiments, a synthetic guide RNA is provided as two separate RNAs where one RNA represents a crRNA segment: 5′-X1-X2-3′ where X1 is the targeting sequence that binds to a portion of a TRIM28 nucleic acid sequence, X2 is a stem sequence the hybridizes to a tracrRNA, and one RNA represents a tracrRNA segment, Z, that is a separate RNA from the crRNA segment and comprises a nucleotide sequence that is partially or completely complementary to X2 of the crRNA.

Exemplary crRNA stem sequences and tracrRNA sequences are provided, for example, in WO/2015/089465, which is incorporated by reference herein. In general, a stem sequence includes any sequence that has sufficient complementarity with a complementary sequence in the tracrRNA to promote formation of a CRISPR complex at a target sequence, wherein the CRISPR complex comprises the stem sequence hybridized to the tracrRNA. In general, degree of complementarity is with reference to the optimal alignment of the stem and complementary sequence in the tracrRNA, along the length of the shorter of the two sequences. Optimal alignment may be determined by any suitable alignment algorithm, and may further account for secondary structures, such as self-complementarity within either the stem sequence or the complementary sequence in the tracrRNA. In some embodiments, the degree of complementarity between the stem sequence and the complementary sequence in the tracrRNA along the length of the shorter of the two when optimally aligned is about or more than about 25%, 30%, 40%, 50%, 60%, 70%, 80%, 90%, 95%, 97.5%, 99%, or higher. In some embodiments, the stem sequence is about or more than about 5, 6, 7, 8, 9, 10, 11, 12, 13, 14, 15, 16, 17, 18, 19, 20, 25, 30, 40, 50, or more nucleotides in length. In some embodiments, the stem sequence and complementary sequence in the tracrRNA are contained within a single RNA, such that hybridization between the two produces a transcript having a secondary structure, such as a hairpin. In some embodiments, the tracrRNA has additional complementary sequences that form hairpins. In some embodiments, the tracrRNA has at least two or more hairpins. In some embodiments, the tracrRNA has two, three, four or five hairpins. In some embodiments, the tracrRNA has at most five hairpins.

In a hairpin structure, the portion of the sequence 5′ of the final “N” and upstream of the loop corresponds to the crRNA stem sequence, and the portion of the sequence 3′ of the loop corresponds to the tracrRNA sequence. Further non-limiting examples of single polynucleotides comprising a guide sequence, a stem sequence, and a tracr sequence are as follows (listed 5′ to 3′), where “N” represents a base of a guide sequence (e.g. a modified oligonucleotide provided herein), the first block of lower case letters represent stem sequence, and the second block of lower case letters represent the tracrRNA sequence, and the final poly-T sequence represents the transcription terminator: (a) NNNNNNNNNNNNNNNNNNNNgtttttgtactctcaagatttaGAAAtaaatcttgcagaagctacaaagataa ggcttcatgccgaaatcaacaccctgtcattttatggcagggtgttttcgttatttaaTTTTTT (SEQ ID NO: 15); (b) NNNNNNNNNNNNNNNNNNNNgtttttgtactctcaGAAAtgcagaagctacaaagataaggcttcatgccg aaatcaacaccctgtcattttatggcagggtgttttcgttatttaaTTTTTT (SEQ ID NO: 16); (c) NNNNNNNNNNNNNNNNNNNNgtttttgtactctcaGAAAtgcagaagctacaaagataaggcttcatgccg aaatcaacaccctgtcattttatggcagggtgtTTTTTT (SEQ ID NO: 17); (d) NNNNNNNNNNNNNNNNNNNNgttttagagctaGAAAtagcaagttaaaataaggctagtccgttatcaactt gaaaaagtggcaccgagtcggtgcTTTTTT (SEQ ID NO: 18); (e) NNNNNNNNNNNNNNNNNNNNgttttagagctaGAAATAGcaagttaaaataaggctagtccgttatcaac ttgaaaaagtgTTTTTTT (SEQ ID NO: 19); and (f) NNNNNNNNNNNNNNNNNNNNgttttagagctagAAATAGcaagttaaaataaggctagtccgttatcaTT TTTTTT (SEQ ID NO: 20).

Selection of suitable oligonucleotides for use in as a targeting sequence in a CRISPR Cas system depends on several factors including the particular CRISPR enzyme to be used and the presence of corresponding proto-spacer adjacent motifs (PAMs) downstream of the target sequence in the target nucleic acid. The PAM sequences direct the cleavage of the target nucleic acid by the CRISPR enzyme. In some embodiments, a suitable PAM is 5′-NRG or 5′-NNGRR (where N is any Nucleotide) for SpCas9 or SaCas9 enzymes (or derived enzymes), respectively. Generally the PAM sequences should be present between about 1 to about 10 nucleotides of the target sequence to generate efficient cleavage of the target nucleic acid. Thus, when the guide RNA forms a complex with the CRISPR enzyme, the complex locates the target and PAM sequence, unwinds the DNA duplex, and the guide RNA anneals to the complementary sequence on the opposite strand. This enables the Cas9 nuclease to create a double-strand break.

A variety of CRISPR enzymes are available for use in conjunction with the disclosed guide RNAs of the present disclosure. In some embodiments, the CRISPR enzyme is a Type II CRISPR enzyme. In some embodiments, the CRISPR enzyme catalyzes DNA cleavage. In some embodiments, the CRISPR enzyme catalyzes RNA cleavage. In some embodiments, the CRISPR enzyme is any Cas9 protein, for instance any naturally-occurring bacterial Cas9 as well as any chimeras, mutants, homologs or orthologs. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified variants thereof. In some embodiments, the CRISPR enzyme cleaves both strands of the target nucleic acid at the Protospacer Adjacent Motif (PAM) site. In some embodiments, the CRISPR enzyme is a nickase, which cleaves only one strand of the target nucleic acid.

Exemplary TRIM28 inhibitory nucleic acid sequences of the present technology, include, but are not limited to: 5′ TTACAGTAGACTGTTCGCTCTC 3′ (SEQ ID NO: 1), 5′ TTCTGCACATCAGACACCTGGC 3′ (SEQ ID NO: 2), 5′ TTGAACTGTTTGAACATGCGGC 3′ (SEQ ID NO: 3), 5′ TTAACTTGTTGAATTGCTTGAA 3′ (SEQ ID NO: 4), 5′ TTCAATAACAATAAGGTTGTAG 3′ (SEQ ID NO: 5), 5′ TAGATCAACTTCTTAGAAAGCA 3′ (SEQ ID NO: 6), 5′ CTACAGGCCGAGTGCAAACA 3′ (SEQ ID NO: 7), 5′ GAGAGCGCCTGCGACCCGAG 3′ (SEQ ID NO: 8), 5′ CCAGCGGGTGAAGTACACCA 3′ (SEQ ID NO: 9), and 5′ CCCAGCCACCAGCTACTGTG 3′ (SEQ ID NO: 10).

### Therapeutic Methods

The following discussion is presented by way of example only, and is not intended to be limiting.

One aspect of the present technology includes methods of treating a disease or condition characterized by elevated expression levels and/or increased activity of TRIM28. Additionally or alternatively, in some embodiments, the present technology includes methods of treating AML. In another aspect, the present disclosure provides a method for inhibiting leukemic cell proliferation in a subject in need thereof, comprising administering to the subject a therapeutically effective amount of at least one Trim28-specific inhibitory nucleic acid, wherein the subject suffers from a disease or condition characterized by elevated expression levels and/or increased activity of TRIM28.

In some embodiments, the subject is diagnosed as having, suspected as having, or at risk of having a disease or condition characterized by elevated expression levels and/or increased activity of TRIM28. Additionally or alternatively, in some embodiments, the subject is diagnosed as having AML.

In therapeutic applications, compositions or medicaments comprising a Trim28-specific inhibitory nucleic acid disclosed herein are administered to a subject suspected of, or already suffering from such a disease or condition (such as, a subject diagnosed with a disease or condition characterized by elevated expression levels and/or increased activity of TRIM28 and/or a subject diagnosed with AML), in an amount sufficient to cure, or at least partially arrest, the symptoms of the disease, including its complications and intermediate pathological phenotypes in development of the disease.

Subjects suffering from a disease or condition characterized by elevated expression levels and/or increased activity of TRIM28 and/or a subject diagnosed with AML can be identified by any or a combination of diagnostic or prognostic assays known in the art. For example, typical symptoms of AML include, but are not limited to, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite.

In some embodiments, the subject may exhibit one or more point mutations in NRAS, DNMT3A, FLT3, KIT, IDH1, IDH2, CEBPA, and NPM1 and/or one or more chromosomal alterations (e.g., an inversion, translocation, or gene fusion) such as CBFB-MYH11, DEK-NUP214, MLL-MLLT3, PML-RARA, RBM15-MKL1, RPN1-EVI1 and RUNX1-RUNX1T1, and are detectable using techniques known in the art. See Naoe & Kiyoi, Int J Hematol. 97(2):165-74 (2013); Shih et al., Nat Rev Cancer. 12(9):599-612 (2012).

In some embodiments, subjects with a disease or condition characterized by elevated expression levels and/or increased activity of TRIM28, and/or subjects suffering from AML that are treated with the Trim28-specific inhibitory nucleic acid will show amelioration or elimination of one or more of the following symptoms: enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite.

In certain embodiments, subjects with a disease or condition characterized by elevated expression levels and/or increased activity of TRIM28, and/or subjects suffering from AML that are treated with the Trim28-specific inhibitory nucleic acid will show reduced leukemic cell proliferation and/or increased survival compared to untreated AML subjects. In certain embodiments, subjects with a disease or condition characterized by elevated expression levels and/or increased activity of TRIM28, and/or subjects suffering from AML that are treated with the Trim28-specific inhibitory nucleic acid will show reduced TRIM28 and H3K9 trimethylation levels compared to untreated AML subjects.

In one aspect, the present disclosure provides a method for monitoring the therapeutic efficacy of a Trim28-specific inhibitory nucleic acid in a subject diagnosed with AML comprising: (a) detecting H3K9 trimethylation levels or Trim28 expression levels in a test sample obtained from the subject after the subject has been administered the Trim28-specific inhibitory nucleic acid; and (b) determining that the Trim28-specific inhibitory nucleic acid is effective when the H3K9 trimethylation levels or Trim28 expression levels in the test sample are reduced compared to that observed in a control sample obtained from the subject prior to administration of the Trim28-specific inhibitory nucleic acid. In some embodiments, H3K9 trimethylation levels are detected via chromatin immunoprecipitation. The test sample may be tissues, cells or biological fluids (blood, plasma, saliva, urine, serum etc.) present within a subject. The Trim28-specific inhibitory nucleic acid may be an antisense oligonucleotide, a shRNA or a sgRNA.

Additionally or alternatively, in some embodiments of the methods disclosed herein, the Trim28-specific inhibitory nucleic acid is complementary to a Trim28 protein domain, such as the RBCC domain, HP1 protein binding domain (HP1BD), Plant Homeodomain (PHD) and Bromodomain. Additionally or alternatively, in some embodiments, the at least one Trim28-specific inhibitory nucleic acid is a sgRNA or shRNA comprising a nucleic acid sequence selected from the group consisting of: 5′ TTACAGTAGACTGTTCGCTCTC 3′ (SEQ ID NO: 1), 5′ TTCTGCACATCAGACACCTGGC 3′ (SEQ ID NO: 2), 5′ TTGAACTGTTTGAACATGCGGC 3′ (SEQ ID NO: 3), 5′ TTAACTTGTTGAATTGCTTGAA 3′ (SEQ ID NO: 4), 5′ TTCAATAACAATAAGGTTGTAG 3′ (SEQ ID NO: 5), 5′ TAGATCAACTTCTTAGAAAGCA 3′ (SEQ ID NO: 6), 5′ CTACAGGCCGAGTGCAAACA 3′ (SEQ ID NO: 7), 5′ GAGAGCGCCTGCGACCCGAG 3′ (SEQ ID NO: 8), 5′ CCAGCGGGTGAAGTACACCA 3′ (SEQ ID NO: 9), and 5′ CCCAGCCACCAGCTACTGTG 3′ (SEQ ID NO: 10).

### Prophylactic Methods

In one aspect, the present technology provides a method for preventing or delaying the onset of a disease or condition characterized by elevated expression levels and/or increased activity of TRIM28. Additionally or alternatively, in some aspects, the present technology provides a method for preventing or delaying the onset AML.

Subjects at risk or susceptible to a disease or condition characterized by elevated expression levels and/or increased activity of TRIM28, and/or subjects at risk or susceptible to AML include those that exhibit one or more point mutations in NRAS, DNMT3A, FLT3, KIT, IDH1, IDH2, CEBPA, and NPM1 and/or one or more chromosomal alterations (e.g., an inversion, translocation, or gene fusion) such as CBFB-MYH11, DEK-NUP214, MLL-MLLT3, PML-RARA, RBM15-MKL1, RPN1-EVI1 and RUNX1-RUNX1T1. Such subjects can be identified by, e.g., any or a combination of diagnostic or prognostic assays known in the art.

In prophylactic applications, pharmaceutical compositions or medicaments comprising a Trim28-specific inhibitory nucleic acid disclosed herein are administered to a subject susceptible to, or otherwise at risk of a disease or condition characterized by elevated expression levels and/or increased activity of TRIM28, and/or a subject susceptible to, or otherwise at risk of AML, in an amount sufficient to eliminate or reduce the risk, or delay the onset of the disease, including biochemical, histologic and/or behavioral symptoms of the disease, its complications and intermediate pathological phenotypes presenting during development of the disease. Administration of a prophylactic Trim28-specific inhibitory nucleic acid can occur prior to the manifestation of symptoms characteristic of the disease or disorder, such that the disease or disorder is prevented or, alternatively, delayed in its progression.

In some embodiments, treatment with the Trim28-specific inhibitory nucleic acid will prevent or delay the onset of one or more of the following symptoms: leukemic cell proliferation, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, and loss of appetite. In certain embodiments, (a) subjects with a disease or condition characterized by elevated expression levels and/or increased activity of TRIM28 and/or (b) subjects with AML that are treated with the Trim28-specific inhibitory nucleic acid will show TRIM28 and/or H3K9 trimethylation levels that resemble those observed in healthy control subjects.

For therapeutic and/or prophylactic applications, a composition comprising a Trim28-specific inhibitory nucleic acid disclosed herein, is administered to the subject. In some embodiments, the Trim28-specific inhibitory nucleic acid is administered one, two, three, four, or five times per day. In some embodiments, the Trim28-specific inhibitory nucleic acid is administered more than five times per day. Additionally or alternatively, in some embodiments, the Trim28-specific inhibitory nucleic acid is administered every day, every other day, every third day, every fourth day, every fifth day, or every sixth day. In some embodiments, the Trim28-specific inhibitory nucleic acid is administered weekly, bi-weekly, tri-weekly, or monthly. In some embodiments, the Trim28-specific inhibitory nucleic acid is administered for a period of one, two, three, four, or five weeks. In some embodiments, the Trim28-specific inhibitory nucleic acid is administered for six weeks or more. In some embodiments, the Trim28-specific inhibitory nucleic acid is administered for twelve weeks or more. In some embodiments, the Trim28-specific inhibitory nucleic acid is administered for a period of less than one year. In some embodiments, the Trim28-specific inhibitory nucleic acid is administered for a period of more than one year. In some embodiments, the Trim28-specific inhibitory nucleic acid is administered throughout the subject's life.

In some embodiments of the methods of the present technology, the Trim28-specific inhibitory nucleic acid is administered daily for 1 week or more. In some embodiments of the methods of the present technology, the Trim28-specific inhibitory nucleic acid is administered daily for 2 weeks or more. In some embodiments of the methods of the present technology, the Trim28-specific inhibitory nucleic acid is administered daily for 3 weeks or more. In some embodiments of the methods of the present technology, the Trim28-specific inhibitory nucleic acid is administered daily for 4 weeks or more. In some embodiments of the methods of the present technology, the Trim28-specific inhibitory nucleic acid is administered daily for 6 weeks or more. In some embodiments of the methods of the present technology, the Trim28-specific inhibitory nucleic acid is administered daily for 12 weeks or more. In some embodiments, the Trim28-specific inhibitory nucleic acid is administered daily throughout the subject's life.

### Determination of the Biological Effect of Trim28-Specific Inhibitory Nucleic Acids

In various embodiments, suitable in vitro or in vivo assays are performed to determine the effect of a specific Trim28-specific inhibitory nucleic acid and whether its administration is indicated for treatment. In various embodiments, in vitro assays can be performed with representative animal models, to determine if a given Trim28-specific inhibitory nucleic acid exerts the desired effect on reducing or eliminating signs and/or symptoms of AML. Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects. In some embodiments, in vitro or in vivo testing is directed to the biological function of one or more Trim28-specific inhibitory nucleic acids.

Animal models of AIL may be generated using techniques known in the art. Such models may be used to demonstrate the biological effect of Trim28-specific inhibitory nucleic acids in the prevention and treatment of conditions arising from disruption of TRIM28, and for determining what comprises a therapeutically effective amount of the one or more Trim28-specific inhibitory nucleic acids disclosed herein in a given context.

### Modes of Administration and Effective Dosages

Any method known to those in the art for contacting a cell, organ or tissue with one or more Trim28-specific inhibitory nucleic acids disclosed herein may be employed. Suitable methods include in vitro, ex vivo, or in vivo methods. In vivo methods typically include the administration of one or more Trim28-specific inhibitory nucleic acids to a mammal, suitably a human. When used in vivo for therapy, the one or more Trim28-specific inhibitory nucleic acids described herein are administered to the subject in effective amounts (i.e., amounts that have desired therapeutic effect). The dose and dosage regimen will depend upon the degree of the disease state of the subject, the characteristics of the particular Trim28-specific inhibitory nucleic acid used, e.g., its therapeutic index, and the subject's history.

The effective amount may be determined during pre-clinical trials and clinical trials by methods familiar to physicians and clinicians. An effective amount of one or more Trim28-specific inhibitory nucleic acids useful in the methods may be administered to a mammal in need thereof by any of a number of well-known methods for administering pharmaceutical compounds. The inhibitors may be administered systemically or locally.

The one or more Trim28-specific inhibitory nucleic acids described herein can be incorporated into pharmaceutical compositions for administration, singly or in combination, to a subject for the treatment or prevention of AML. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

Pharmaceutical compositions are typically formulated to be compatible with its intended route of administration. Examples of routes of administration include parenteral (e.g., intravenous, intradermal, intraperitoneal or subcutaneous), oral, inhalation, transdermal (topical), intraocular, iontophoretic, and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic. For convenience of the patient or treating physician, the dosing formulation can be provided in a kit containing all necessary equipment (e.g., vials of drug, vials of diluent, syringes and needles) for a treatment course (e.g., 7 days of treatment).

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, CREMOPHOR EL™ (BASF, Parsippany, N.J.) or phosphate buffered saline (PBS). In all cases, a composition for parenteral administration must be sterile and should be fluid to the extent that easy syringability exists. It should be stable under the conditions of manufacture and storage and must be preserved against the contaminating action of microorganisms such as bacteria and fungi.

The pharmaceutical compositions having one or more Trim28-specific inhibitory nucleic acids disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. Glutathione and other antioxidants can be included to prevent oxidation. In many cases, it will be advantageous to include isotonic agents, for example, sugars, polyalcohols such as mannitol, sorbitol, or sodium chloride in the composition. Prolonged absorption of the injectable compositions can be brought about by including in the composition an agent that delays absorption, for example, aluminum monostearate or gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, typical methods of preparation include vacuum drying and freeze drying, which can yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressurized container or dispenser, which contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays. For transdermal administration, the active compounds are formulated into ointments, salves, gels, or creams as generally known in the art. In one embodiment, transdermal administration may be performed by iontophoresis.

A therapeutic agent can be formulated in a carrier system. The carrier can be a colloidal system. The colloidal system can be a liposome, a phospholipid bilayer vehicle. In one embodiment, the therapeutic agent is encapsulated in a liposome while maintaining the agent's structural integrity. One skilled in the art would appreciate that there are a variety of methods to prepare liposomes. (See Lichtenberg, et al., Methods Biochem. Anal., 33:337-462 (1988); Anselem, et al., Liposome Technology, CRC Press (1993)). Liposomal formulations can delay clearance and increase cellular uptake (See Reddy, Ann. Pharmacother., 34(7-8):915-923 (2000)). An active agent can also be loaded into a particle prepared from pharmaceutically acceptable ingredients including, but not limited to, soluble, insoluble, permeable, impermeable, biodegradable or gastroretentive polymers or liposomes. Such particles include, but are not limited to, nanoparticles, biodegradable nanoparticles, microparticles, biodegradable microparticles, nanospheres, biodegradable nanospheres, microspheres, biodegradable microspheres, capsules, emulsions, liposomes, micelles and viral vector systems.

The carrier can also be a polymer, e.g., a biodegradable, biocompatible polymer matrix. In one embodiment, the therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., collagen, fibronectin, elastin, cellulose acetate, cellulose nitrate, polysaccharide, fibrin, gelatin, and combinations thereof. In one embodiment, the polymer is poly-lactic acid (PLA) or copoly lactic/glycolic acid (PGLA). The polymeric matrices can be prepared and isolated in a variety of forms and sizes, including microspheres and nanospheres. Polymer formulations can lead to prolonged duration of therapeutic effect. (See Reddy, Ann. Pharmacother., 34(7-8):915-923 (2000)). A polymer formulation for human growth hormone (hGH) has been used in clinical trials. (See Kozarich and Rich, Chemical Biology, 2:548-552 (1998)).

Examples of polymer microsphere sustained release formulations are described in PCT publication WO 99/15154 (Tracy, et al.), U.S. Pat. Nos. 5,674,534 and 5,716,644 (both to Zale, et al.), PCT publication WO 96/40073 (Zale, et al.), and PCT publication WO 00/38651 (Shah, et al.). U.S. Pat. Nos. 5,674,534 and 5,716,644 and PCT publication WO 96/40073 describe a polymeric matrix containing particles of erythropoietin that are stabilized against aggregation with a salt.

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using known techniques. The materials can also be obtained commercially, e.g., from Alza Corporation and Nova Pharmaceuticals, Inc. Liposomal suspensions (including liposomes targeted to specific cells with monoclonal antibodies to cell-specific antigens) can also be used as pharmaceutically acceptable carriers. These can be prepared according to methods known to those skilled in the art, for example, as described in U.S. Pat. No. 4,522,811.

The therapeutic compounds can also be formulated to enhance intracellular delivery. For example, liposomal delivery systems are known in the art, see, e.g., Chonn and Cullis, “Recent Advances in Liposome Drug Delivery Systems,” Current Opinion in Biotechnology 6:698-708 (1995); Weiner, “Liposomes for Protein Delivery: Selecting Manufacture and Development Processes,” Immunomethods, 4(3):201-9 (1994); and Gregoriadis, “Engineering Liposomes for Drug Delivery: Progress and Problems,” Trends Biotechnol., 13(12):527-37 (1995). Mizguchi, et al., Cancer Lett., 100:63-69 (1996), describes the use of fusogenic liposomes to deliver a protein to cells both in vivo and in vitro.

Dosage, toxicity and therapeutic efficacy of any therapeutic agent can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are advantageous. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately. Levels in plasma may be measured, for example, by high performance liquid chromatography.

Typically, an effective amount of the one or more Trim28-specific inhibitory nucleic acids disclosed herein sufficient for achieving a therapeutic or prophylactic effect, range from about 0.000001 mg per kilogram body weight per day to about 10,000 mg per kilogram body weight per day. Suitably, the dosage ranges are from about 0.0001 mg per kilogram body weight per day to about 100 mg per kilogram body weight per day. For example dosages can be 1 mg/kg body weight or 10 mg/kg body weight every day, every two days or every three days or within the range of 1-10 mg/kg every week, every two weeks or every three weeks. In one embodiment, a single dosage of the therapeutic compound ranges from 0.001-10,000 micrograms per kg body weight. In one embodiment, one or more Trim28-specific inhibitory nucleic acid concentrations in a carrier range from 0.2 to 2000 micrograms per delivered milliliter. An exemplary treatment regime entails administration once per day or once a week. In therapeutic applications, a relatively high dosage at relatively short intervals is sometimes required until progression of the disease is reduced or terminated, or until the subject shows partial or complete amelioration of symptoms of disease. Thereafter, the patient can be administered a prophylactic regime.

In some embodiments, a therapeutically effective amount of one or more Trim28-specific inhibitory nucleic acids may be defined as a concentration of inhibitor at the target tissue of 10⁻³² to 10⁻⁶ molar, e.g., approximately 10⁻⁷ molar. This concentration may be delivered by systemic doses of 0.001 to 100 mg/kg or equivalent dose by body surface area. The schedule of doses would be optimized to maintain the therapeutic concentration at the target tissue, such as by single daily or weekly administration, but also including continuous administration (e.g., parenteral infusion or transdermal application).

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

The mammal treated in accordance with the present methods can be any mammal, including, for example, farm animals, such as sheep, pigs, cows, and horses; pet animals, such as dogs and cats; laboratory animals, such as rats, mice and rabbits. In some embodiments, the mammal is a human.

### Combination Therapy

In some embodiments, one or more Trim28-specific inhibitory nucleic acids disclosed herein may be combined with one or more additional therapies for the prevention or treatment of AML. Additional therapeutic agents include, but are not limited to, chemotherapeutic agents, arsenic trioxide (Trisenox), all-trans retinoic acid (ATRA), and stem cell transplants.

In some embodiments, the one or more Trim28-specific inhibitory nucleic acids disclosed herein may be separately, sequentially or simultaneously administered with at least one additional therapeutic agent selected from the group consisting of alkylating agents, topoisomerase inhibitors, endoplasmic reticulum stress inducing agents, antimetabolites, mitotic inhibitors, nitrogen mustards, nitrosoureas, alkylsulfonates, platinum agents, taxanes, vinca agents, anti-estrogen drugs, aromatase inhibitors, ovarian suppression agents, VEGF/VEGFR inhibitors, EGF/EGFR inhibitors, PARP inhibitors, cytostatic alkaloids, cytotoxic antibiotics, antimetabolites, endocrine/hormonal agents, bisphosphonate therapy agents, phenphormin and targeted biological therapy agents (e.g., therapeutic peptides described in U.S. Pat. No. 6,306,832, WO 2012007137, WO 2005000889, WO 2010096603 etc.). In some embodiments, the at least one additional therapeutic agent is a chemotherapeutic agent.

Specific chemotherapeutic agents include, but are not limited to, cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, or combinations thereof.

Examples of antimetabolites include 5-fluorouracil (5-FU), 6-mercaptopurine (6-MP), capecitabine, cytarabine, floxuridine, fludarabine, gemcitabine, hydroxyurea, methotrexate, pemetrexed, and mixtures thereof.

Examples of taxanes include accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, and mixtures thereof.

Examples of DNA alkylating agents include cyclophosphamide, chlorambucil, melphalan, bendamustine, uramustine, estramustine, carmustine, lomustine, nimustine, ranimustine, streptozotocin; busulfan, mannosulfan, and mixtures thereof.

Examples of topoisomerase I inhibitor include SN-38, ARC, NPC, camptothecin, topotecan, 9-nitrocamptothecin, exatecan, lurtotecan, lamellarin D9-aminocamptothecin, rubifen, gimatecan, diflomotecan, BN80927, DX-8951f, MAG-CPT, and mixtures thereof. Examples of topoisomerase II inhibitors include amsacrine, etoposide, etoposide phosphate, teniposide, daunorubicin, mitoxantrone, amsacrine, ellipticines, aurintricarboxylic acid, doxorubicin, and HU-331 and combinations thereof.

In certain embodiments, an additional therapeutic agent is administered to a subject in combination with the one or more Trim28-specific inhibitory nucleic acids disclosed herein such that a synergistic therapeutic effect is produced. For example, administration of one or more Trim28-specific inhibitory nucleic acids with one or more additional therapeutic agents for the prevention or treatment of AML will have greater than additive effects in the prevention or treatment of the disease. For example, lower doses of one or more of the therapeutic agents may be used in treating or preventing AML resulting in increased therapeutic efficacy and decreased side-effects. In some embodiments, the one or more Trim28-specific inhibitory nucleic acids disclosed herein are administered in combination with any of the at least one additional therapeutic agents described above, such that a synergistic effect in the prevention or treatment of AML results.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents.

### Kits

The present disclosure also provides kits for the prevention and/or treatment of AML comprising one or more of Trim28-specific inhibitory nucleic acids disclosed herein (e.g., inhibitory nucleic acids comprising a nucleic acid sequence of any one of SEQ ID NOs: 1-10). Optionally, the above described components of the kits of the present technology are packed in suitable containers and labeled for the prevention and/or treatment of AML.

The above-mentioned components may be stored in unit or multi-dose containers, for example, sealed ampoules, vials, bottles, syringes, and test tubes, as an aqueous, preferably sterile, solution or as a lyophilized, preferably sterile, formulation for reconstitution. The kit may further comprise a second container which holds a diluent suitable for diluting the pharmaceutical composition towards a higher volume. Suitable diluents include, but are not limited to, the pharmaceutically acceptable excipient of the pharmaceutical composition and a saline solution. Furthermore, the kit may comprise instructions for diluting the pharmaceutical composition and/or instructions for administering the pharmaceutical composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or diagnostic products, that contain information about, for example, the indications, usage, dosage, manufacture, administration, contraindications and/or warnings concerning the use of such therapeutic or diagnostic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: In Vivo shRNA Screening Identifies Trim28 as an AML Maintenance Gene

To identify potential therapeutic targets in epigenetic pathways, a focused RNAi “drop out” screen was performed using a short hairpin RNA (shRNA) library in a mouse AML model (see J. Zuber et al., Nat Biotechnol 29(1), 79-83 (2011)). A shRNA library specifically targeting these genes was constructed with doxycycline inducible promoters and introduced as a pool into Nras(G12D)/MLL-AF9 leukemic cells. After the AML cells engrafted, shRNAs were induced by doxycycline. The abundance of each shRNA from tumor cells recovered from moribund mice was compared to its abundance in the pre-engrafted cell population. Exemplary shRNAs and sgRNAs used in the Examples described herein are provided below:

| mouse shRNA | Sequence | |
|---|---|---|
| P dxk shRNA 307 | TTATTGACATCGTTCACCTTGA | (SEQ ID NO: 21) |
| P dxk shRNA 3259 | TGTAACCTCACATTGAACCTGA | (SEQ ID NO: 22) |
| Trim28.1180 | TTACAGTAGACTGTTCGCTCTC | (SEQ ID NO: 1) |
| Trim28.1417 | TTCTGCACATCAGACACCTGGC | (SEQ ID NO: 2) |
| Trim28.2866 | TTGAACTGTTTGAACATGCGGC | (SEQ ID NO: 3) |
| human shRNA | Sequence | |
| hTrim28.2594 | TTAACTTGTTGAATTGCTTGAA | (SEQ ID NO: 4) |
| hTrim28.1837 | TTCAATAACAATAAGGTTGTAG | (SEQ ID NO: 5) |
| hTrim28.1374 | TAGATCAACTTCTTAGAAAGCA | (SEQ ID NO: 6) |
| hBRD4-aa437 | GTACAACCCTCCTGACCATG | (SEQ ID NO: 23) |
| hTRIM28-a84 | CTACAGGCCGAGTGCAAACA | (SEQ ID NO: 7) |
| hTRIM28-a75 | GAGAGCGCCTGCGACCCGAG | (SEQ ID NO: 8) |
| hTRIM28-a187 | CCAGCGGGTGAAGTACACCA | (SEQ ID NO: 9) |
| hTRIM28-a166 | CCCAGCCACCAGCTACTGTG | (SEQ ID NO: 10) |

As expected, control shRNAs targeting genes known to be essential for AIL cell proliferation were depleted in the screen (e.g., Cdk9, Brd4) (J. Zuber et al., Genes Dev 25, 1628 (2011)), whereas the abundance of neutral shRNAs (e.g. targeting Renilla luciferase) remained largely unchanged. These observations support the robustness of the screening results. 1000 genes encoding epigenetic regulators were analyzed and 13 genes were found to have more than 3 shRNAs depleted in the bone marrow (BM) tumor harvest (FIGS. 1A-1B).

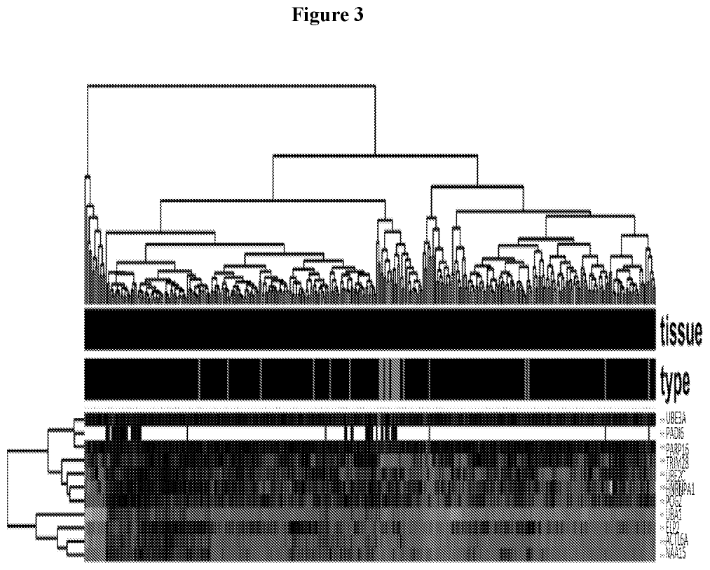

To prioritize the screen hits, publicly available data from a series of genome-wide CRISPR/Cas9 screens in human cancer cells were analyzed (FIG. 3). Some of the hits (NAA15, ELP2, ACTL6A, UBA1) appeared to be universally toxic for all cancer cell lines and are likely to be essential genes. For those that showed selectivity (TRIM28, UBE2C, HNRNPA1, POGZ), the clinical correlation between the expression level of the gene and prognosis in AML patients was checked. Only TRIM28 showed an association between higher expression and worse prognosis (FIG. 1C).

These results demonstrate that the Trim28-specific inhibitory nucleic acids of the present technology are useful in methods for treating or preventing acute myeloid leukemia in a subject in need thereof.

### Example 2: Multiple Mouse AML Cell Lines are Sensitive to Trim28 Suppression In Vitro

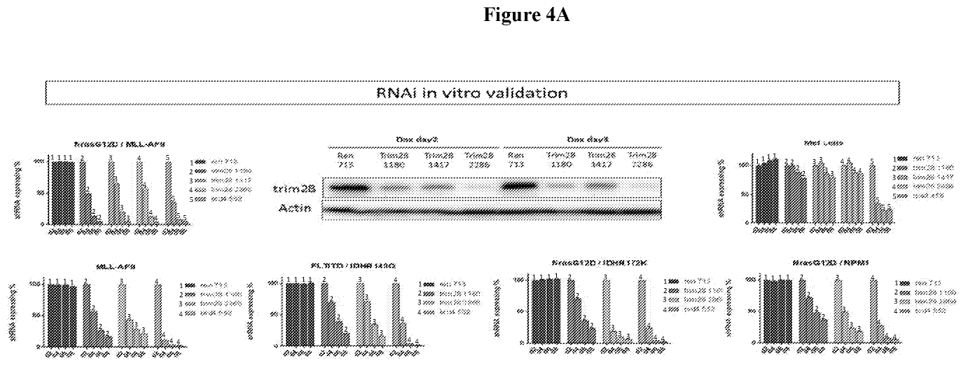

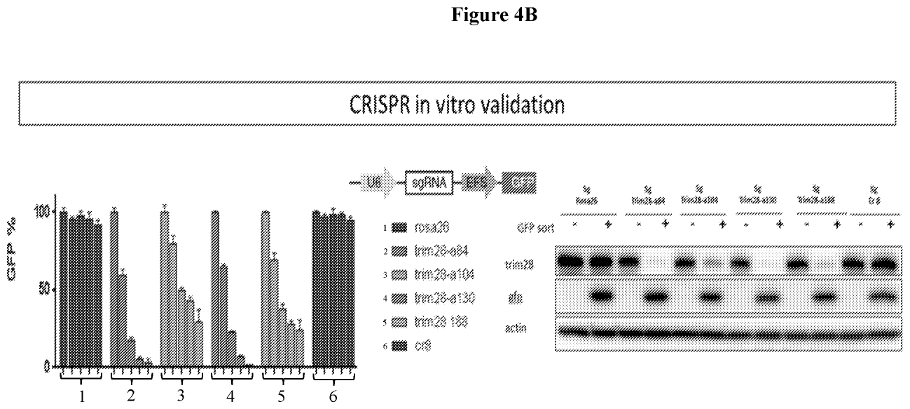

Individual shRNAs that target Trim28 were generated and competition assays were performed in multiple murine AIL cell lines. Consistent with in vivo shRNA screening results described herein, knockdown of Trim28 using individual shRNAs inhibited proliferation of Nras(G12D)/MLL-AF9 leukemia cells, as well as AML cell lines with other genetic drivers (FIG. 4A). The validity of these findings was reinforced by sgRNA-mediated knockout of Trim28 as an orthogonal approach (FIG. 4B). Although the rate at which Trim28 shRNAs was depleted from AML cells was slower than observed for essential genes such as Brd4 or Rpa3, their effects were much more specific to leukemia cells. In contrast to shRNAs targeting Rpa3, Trim28 shRNAs showed modest to no anti-proliferative effects in mouse embryonic fibroblasts (MEF) cells (FIG. 4A).

These results demonstrate that the Trim28-specific inhibitory nucleic acids of the present technology are useful in methods for treating or preventing acute myeloid leukemia in a subject in need thereof.

### Example 3: Mouse AML Cells are Sensitive to Trim28 Suppression In Vivo

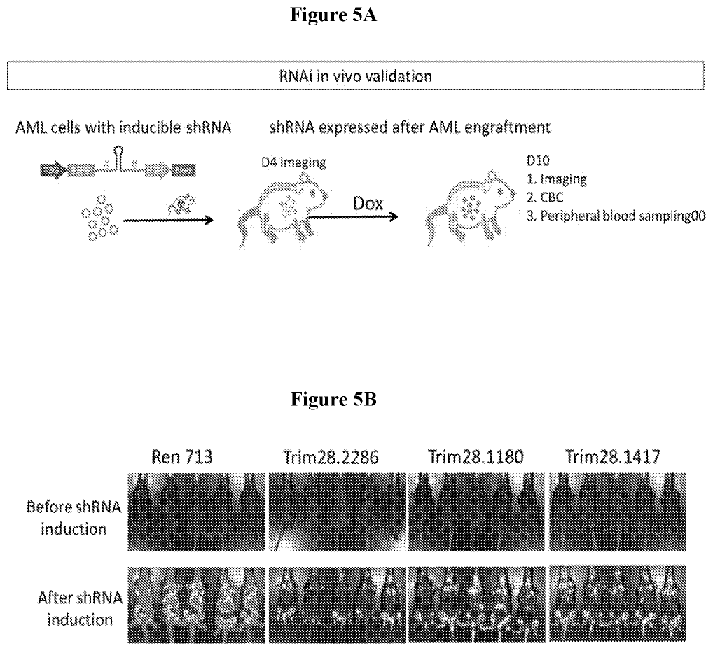

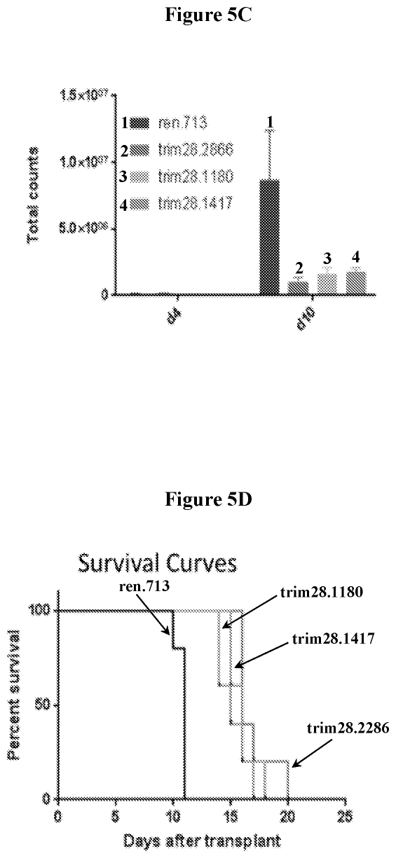

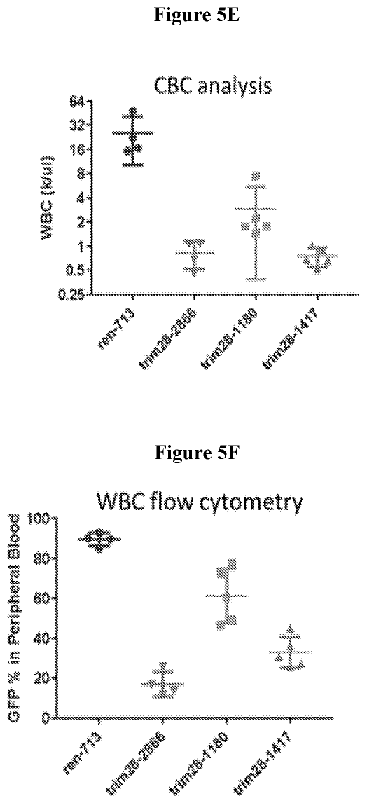

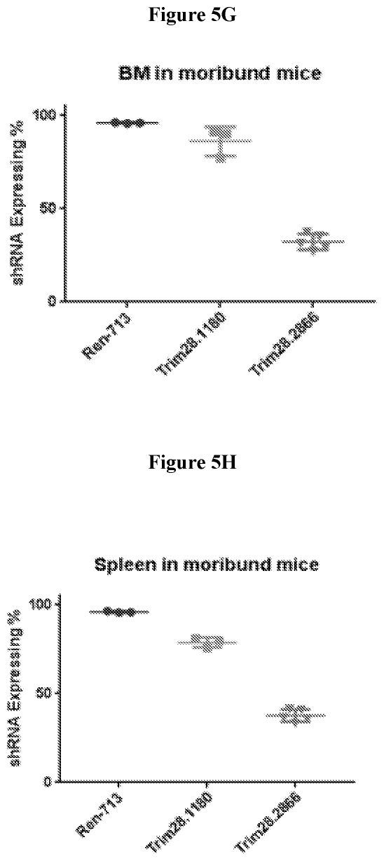

In order to test the in vivo significance of Trim28 for leukemia disease progression, the consequences of suppressing Trim28 in established leukemia in vivo was evaluated using an AML mouse model with a reverse tetracycline transactivator (rtTA) and a tetracycline-responsive element (TRE) promoter, where shRNA expression is induced by doxycycline (J. Zuber et al., Nat Biotechnol 29(1), 79-83 (2011)). In this mouse model, transfusion oncogene MLL-AF9 was co-transcribed with rtTA element; while Nras(G12D) was co-expressed with luciferase to allow for monitoring of disease progression with bioluminescence imaging. These Nras(G12D)/MLL-AF9 leukemic cells were transduced with viruses encoding shRNAs targeting control Renilla luciferase or Trim28, and were subsequently transplanted into sub-lethally irradiated recipient mice (FIG. 5A). Induction of Trim28 shRNAs by doxycycline treatment significantly delayed disease progression, reduced tumor burden in the peripheral blood, and extended overall animal survival (FIG. 5D). Consistent with the decrease of luciferase intensity (FIGS. 5B-5C), the percentage of GFP+ cells in bone marrow was decreased (FIGS. 5E-5H), suggesting that leukemia cells escaping PDXK knockdown or lacking shRNA expression are responsible for progressive disease.

These results demonstrate that the Trim28-specific inhibitory nucleic acids of the present technology are useful in methods for treating or preventing acute myeloid leukemia in a subject in need thereof.

### Example 4: Human AML Lines are Sensitive to TRIM28 Suppression In Vitro

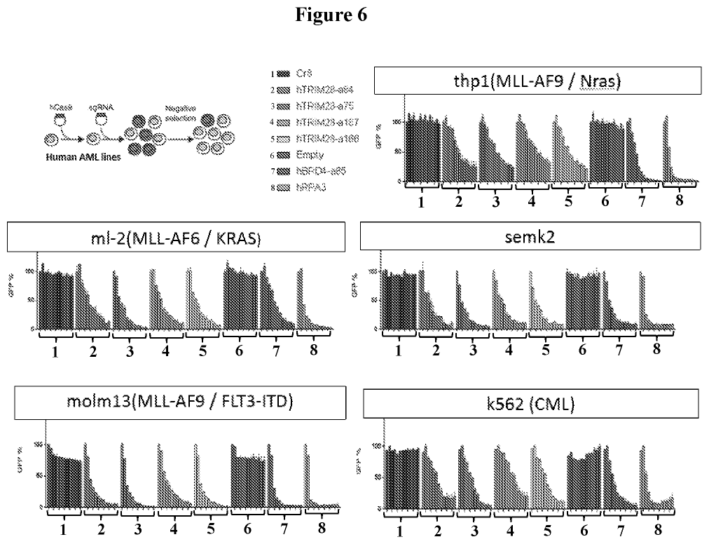

To validate the human relevance of TRIM28, multiple human AML cell lines engineered to harbor constitutively expressed Cas9 and sgRNAs against TRIM28 were tested in vitro (FIG. 6). Over the course of 30 days, TRIM28 sgRNA infected human AML cells (GFP+) were depleted as were cells infected with sgRNAs targeting other cell essential genes (BRD4, RPA3). In contrast, AML cells infected with empty vector or with an sgRNA targeting the mouse genome (CR8) did not show any disadvantage in proliferation.

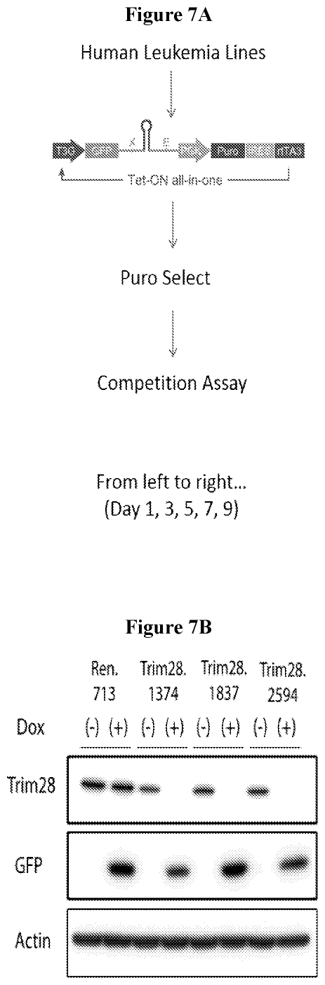

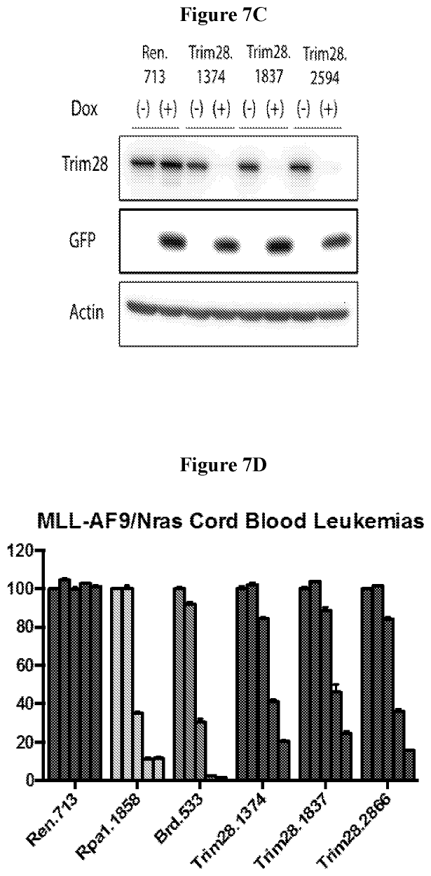

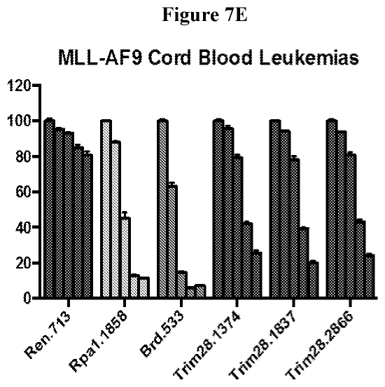

In addition to established AML lines, primary human AMLs generated from cord blood cells were also tested (FIG. 7A). Using a vector construct with Tre3G promoter together with a cis-rtTA element and selection marker, human lines with inducible shRNA expression were generated and selected. The efficacy of shRNA against human TRIM28 was verified by western blotting for shRNA-expressing Molm13/Thp1 cells (FIGS. 7B-7C). Using a fluorescence based competition assay as described before, reduced fitness of TRIM28-suppressed human cord blood derived AML cells in comparison to uninfected parental lines was demonstrated (FIGS. 7D-7E).

These results demonstrate that the Trim28-specific inhibitory nucleic acids of the present technology are useful in methods for treating or preventing acute myeloid leukemia in a subject in need thereof.

### Example 5: Trim28 Inhibition Leads to Myeloid Differentiation and Leukemia Stem-Cell Depletion

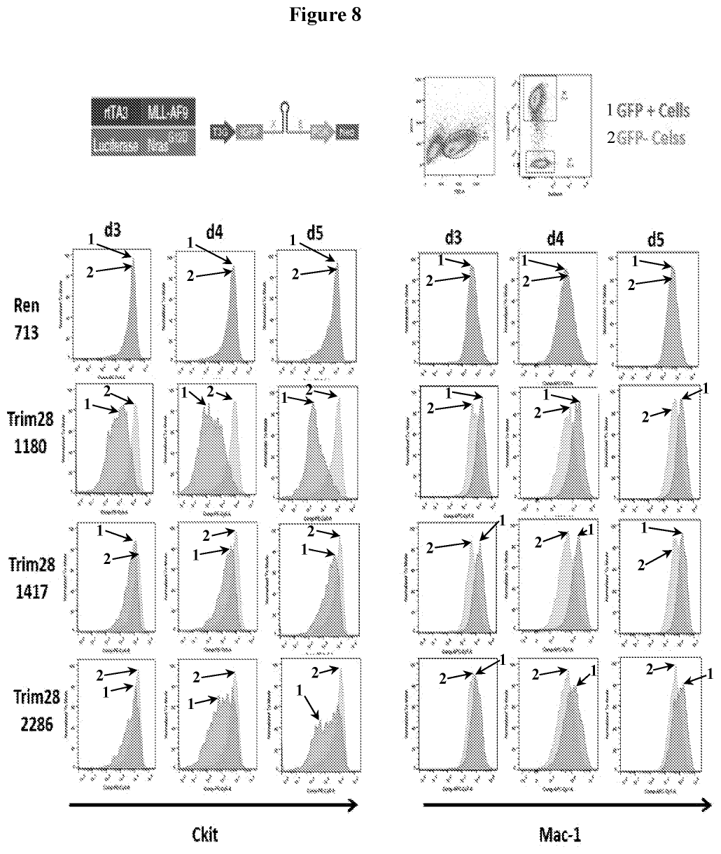

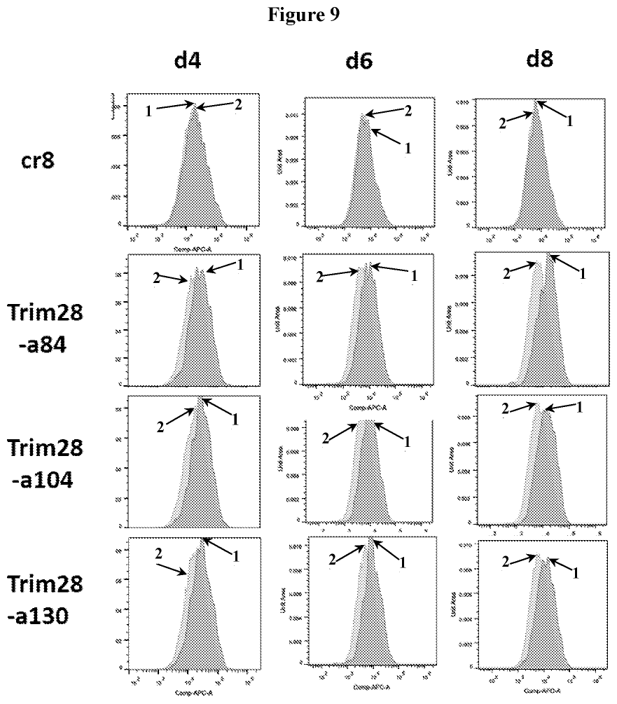

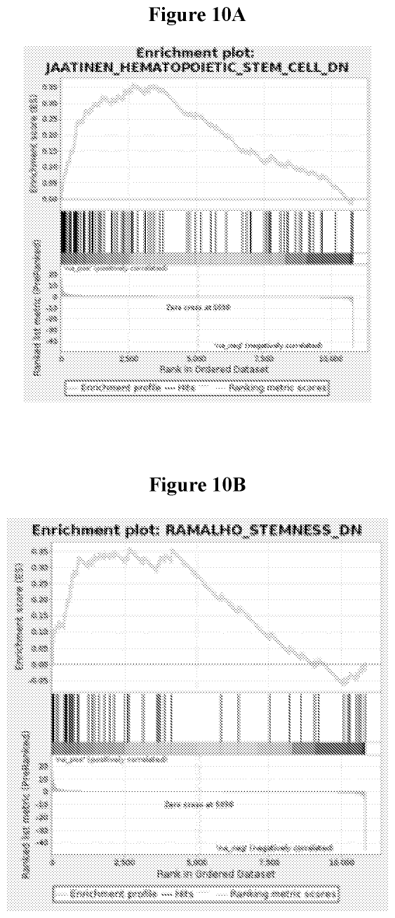

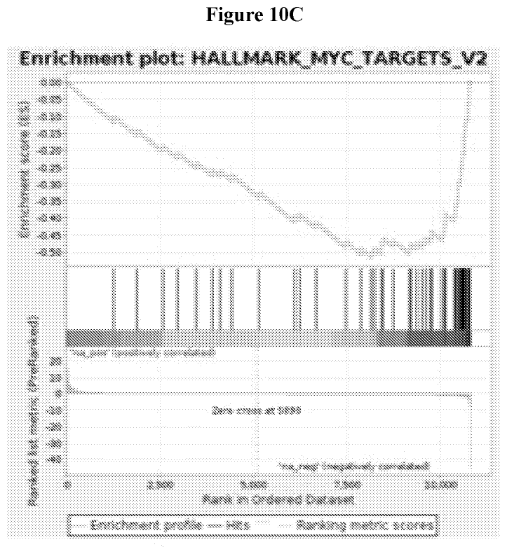

One of the characteristics of AML is an expanded self-renewal capacity together with an inability to complete terminal myeloid differentiation. The effect of Trim28 on the differentiation state of leukemia cells was evaluated. Both expression of Trim28 shRNA and Trim28 sgRNA altered the morphology of MLL-AF9/Nras(G12D) leukemia from myelomonocytic blasts to cells with a macrophage-like appearance. Upon Trim28 inhibition, leukemia cells showed increased surface expression of integrin αM (Mac-1), a myeloid differentiation marker, and decreased expression of c-Kit, a cell surface marker associated with leukemia stem cells (LSCs) in mouse models of MLL-rearranged leukemia (FIG. 8 and FIG. 9). RNA sequencing (RNA-seq) analysis using Trim28 KO MLL-AF9 leukemia cells was also performed. Through cancer hallmark signature analysis and gene set enrichment analysis (GSEA), significant down regulation of Myc targets and upregulation of genes lowly expressed in stem cells was identified (FIG. 10). This finding is consistent with recent evidence demonstrating that the Myc transcriptional network has an important role in LSC self-renewal (J. Kim et al., Cell 143, 313 (2010)).

These results demonstrate that the Trim28-specific inhibitory nucleic acids of the present technology are useful in methods for treating or preventing acute myeloid leukemia in a subject in need thereof.

### Example 6: Possible Mechanisms of Trim28 Dependency

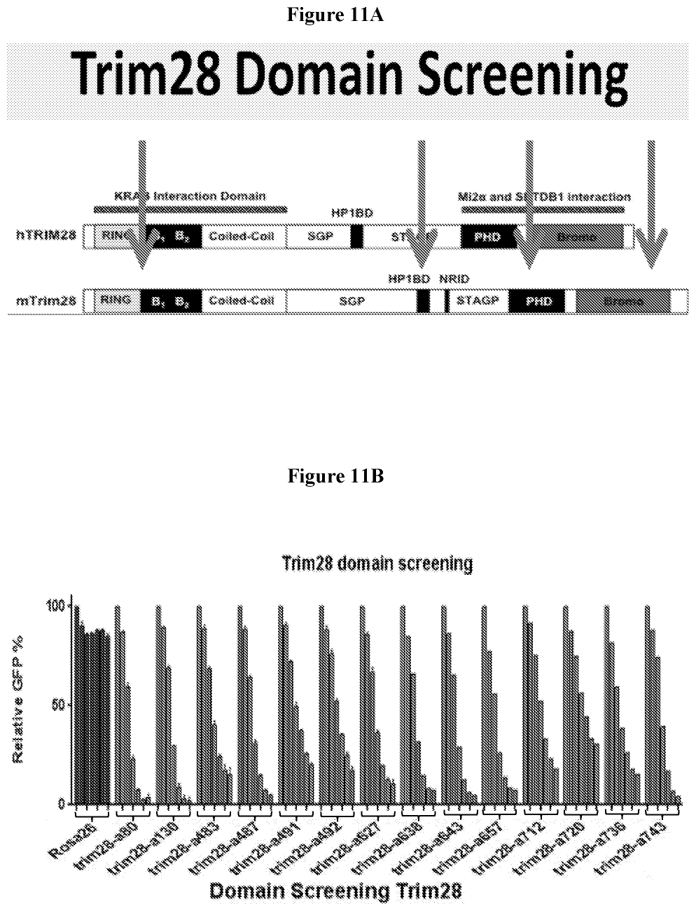

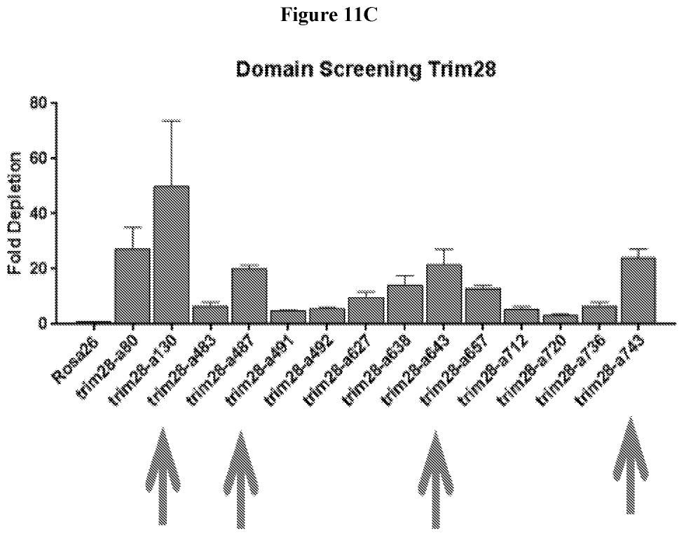

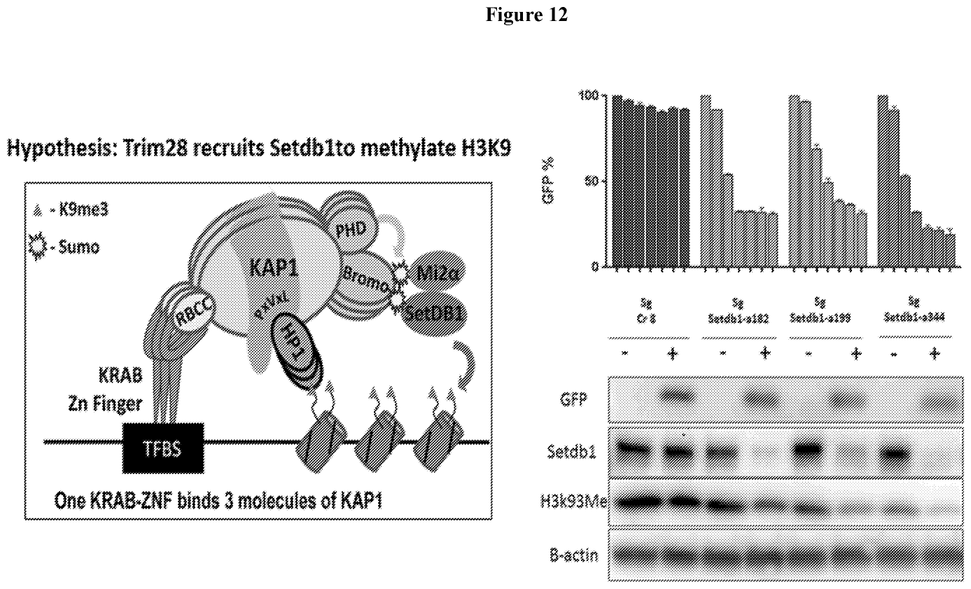

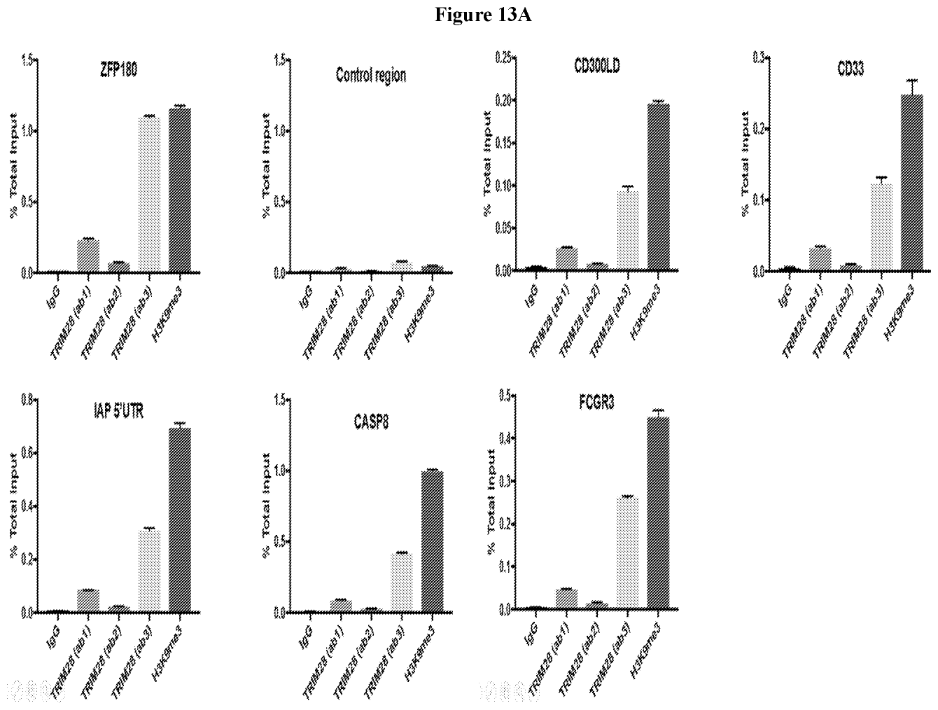

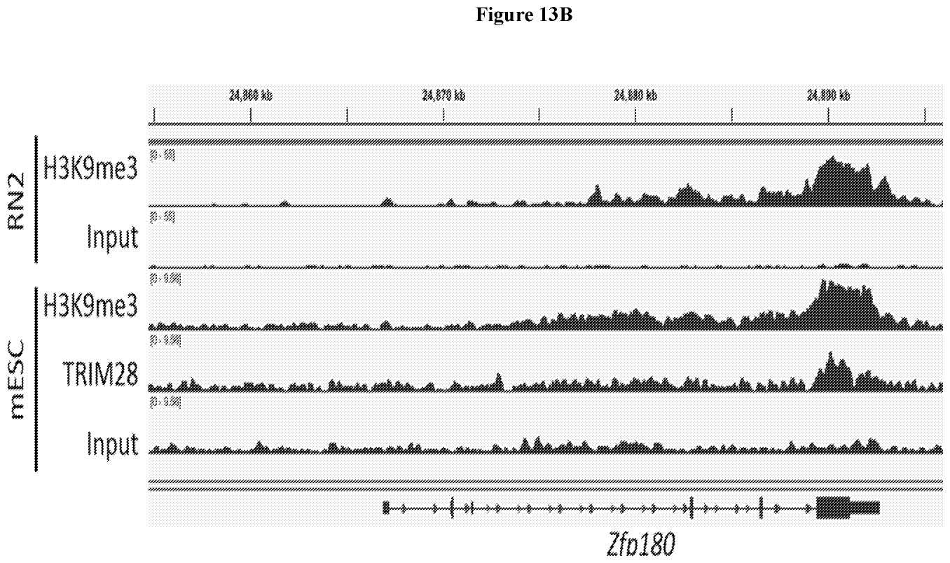

To determine how Trim28 controls acute myeloid leukemia differentiation, a domain-focused CRISPR strategy (see J. Shi et al., Nat Biotechnol 33: 661 (2015)) was utilized. This method used sgRNAs targeting different domains of a target protein and has been successful in interrogating the most critical domains in cancer cell maintenance. sgRNA guides targeting different domains of Trim28 cDNA were cloned and tested for their ability to reduce mouse AML cell fitness. As shown in FIGS. 11A-11C, fold-depletion of each sgRNA was plotted and the KRAB interaction domain and the HP1 binding domain were found to be more sensitive to disruption by their targeting guides. This result is consistent with the hypothesis that Trim28 impacts the AML differentiation program by recruiting chromatin modifying enzyme Setdb1. Setdb1 is a methyltransferase that catalyzes trimethylation of H3K9 (H3K9me3). In embryonic stem cells (ESCs) (S. Koide et al., Blood 128, 638 (2016)) Setdb1 functions as a silencer for endogenous retroviral elements (ERVs). This protein has been shown to be essential in the maintenance of mouse hematopoietic stem and progenitor cells (HSPCs), as well as leukemic cells. To further validate Setdb1 as critical for leukemia cell survival, a Setdb1 knockout experiment was performed as shown in FIG. 12. Suppression of Setdb1 phenocopied the loss of Trim28, reducing the fitness of AML cells. Interestingly, a slight reduction of H3K9 trimethylation was observed globally upon the knockout of Setdb1 (FIG. 12), suggesting that the enzymatic function of Setdb1 may be required for AML maintenance. In MEF cells, published ChIP-seq data show that Trim28 co-localizes with H3K9-trimethylated chromatin in many distinct genetic loci. This co-localization was validated experimentally by ChIP-qPCR at 6 different loci (FIG. 13A). ChIP-seq data of H3K9 trimethylation in mouse leukemia cells was generated, and some overlap between H3K9 trimethylation peaks with mouse embryonic stem cell (mESC) peaks was observed (FIG. 13B), suggesting that Trim28 co-localizes with H3K9 trimethylated genomic loci in leukemia cells.

These results demonstrate that the Trim28-specific inhibitory nucleic acids of the present technology are useful in methods for treating or preventing acute myeloid leukemia in a subject in need thereof.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for treating or preventing acute myeloid leukemia in a subject in need thereof, comprising administering to the subject a therapeutically effective amount of at least one Trim28-specific inhibitory nucleic acid that inhibits Trim28 activity in the subject.

2. The method of claim 1, wherein the at least one Trim28-specific inhibitory nucleic acid is complementary to a Trim28 protein domain selected from the group consisting of RBCC domain, HP1 protein binding domain (HP1BD), Plant Homeodomain (PHD) and Bromodomain.

3. The method of claim 1, wherein the at least one Trim28-specific inhibitory nucleic acid is a sgRNA or shRNA comprising a nucleic acid sequence selected from the group consisting of:
(SEQ ID NO: 1)
5′ TTACAGTAGACTGTTCGCTCTC 3′,
(SEQ ID NO: 2)
5′ TTCTGCACATCAGACACCTGGC 3′,
(SEQ ID NO: 3)
5′ TTGAACTGTTTGAACATGCGGC 3′,
(SEQ ID NO: 4)
5′ TTAACTTGTTGAATTGCTTGAA 3′,
(SEQ ID NO: 5)
5′ TTCAATAACAATAAGGTTGTAG 3′,
(SEQ ID NO: 6)
5′ TAGATCAACTTCTTAGAAAGCA 3′,
(SEQ ID NO: 7)
5′ CTACAGGCCGAGTGCAAACA 3′,
(SEQ ID NO: 8)
5′ GAGAGCGCCTGCGACCCGAG 3′,
(SEQ ID NO: 9)
5′ CCAGCGGGTGAAGTACACCA 3′,
and
(SEQ ID NO: 10)
5′ CCCAGCCACCAGCTACTGTG 3′.

4. The method of claim 1, wherein the subject displays elevated expression levels of Trim28 protein in leukemic cells prior to treatment.

5. The method of claim 1, wherein the subject has been diagnosed as having AML.

6. The method of claim 5, wherein the signs or symptoms of AML comprise one or more of leukemic cell proliferation, enlarged lymph nodes, anemia, neutropenia, leukopenia, leukostasis, chloroma, granulocytic sarcoma, myeloid sarcoma, fatigue, weakness, dizziness, chills, headaches, shortness of breath, thrombocytopenia, excess bruising and bleeding, frequent or severe nosebleeds, bleeding gums, gum pain and swelling, headache, weakness in one side of the body, slurred speech, confusion, sleepiness, blurry vision, vision loss, deep venous thrombosis (DVT), pulmonary embolism, bone or joint pain, swelling in the abdomen, seizures, vomiting, facial numbness, defects in balance, weight loss, fever, night sweats, or loss of appetite.

7. The method of claim 1, wherein the subject harbors one or more point mutations in NRAS, DNMT3A, FLT3, KIT, IDH1, IDH2, CEBPA and NPM1.

8. The method of claim 1, wherein the subject harbors one or more gene fusions selected from the group consisting of CBFB-MYH11, DEK-NUP214, MLL-MLLT3, PML-RARA, RBM15-MKL1, RPN1-EVI1 and RUNX1-RUNX1T1.

9. The method of claim 1, wherein the subject is human.

10. The method of claim 1, wherein the at least one Trim28-specific inhibitory nucleic acid is administered orally, topically, intranasally, systemically, intravenously, subcutaneously, intraperitoneally, intradermally, intraocularly, iontophoretically, transmucosally, or intramuscularly.

11. The method of claim 1, further comprising separately, sequentially or simultaneously administering one or more additional therapeutic agents to the subject.

12. The method of claim 11, wherein the one or more additional therapeutic agents are selected from the group consisting of cyclophosphamide, fluorouracil (or 5-fluorouracil or 5-FU), methotrexate, edatrexate (10-ethyl-10-deaza-aminopterin), thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolmide, topotecan, vincristine, vinblastine, eribulin, mutamycin, capecitabine, anastrozole, exemestane, letrozole, leuprolide, abarelix, buserlin, goserelin, megestrol acetate, risedronate, pamidronate, ibandronate, alendronate, denosumab, zoledronate, trastuzumab, tykerb, anthracyclines (e.g., daunorubicin and doxorubicin), cladribine, midostaurin, bevacizumab, oxaliplatin, melphalan, etoposide, mechlorethamine, bleomycin, microtubule poisons, annonaceous acetogenins, chlorambucil, ifosfamide, streptozocin, carmustine, lomustine, busulfan, dacarbazine, temozolomide, altretamine, 6-mercaptopurine (6-MP), cytarabine, floxuridine, fludarabine, hydroxyurea, pemetrexed, epirubicin, idarubicin, SN-38, ARC, NPC, campothecin, 9-nitrocamptothecin, 9-aminocamptothecin, rubifen, gimatecan, diflomotecan, BN80927, DX-8951f, MAG-CPT, amsacnne, etoposide phosphate, teniposide, azacitidine (Vidaza), decitabine, accatin III, 10-deacetyltaxol, 7-xylosyl-10-deacetyltaxol, cephalomannine, 10-deacetyl-7-epitaxol, 7-epitaxol, 10-deacetylbaccatin III, 10-deacetyl cephalomannine, streptozotocin, nimustine, ranimustine, bendamustine, uramustine, estramustine, mannosulfan, camptothecin, exatecan, lurtotecan, lamellarin D9-aminocamptothecin, amsacrine, ellipticines, aurintricarboxylic acid, HU-331, and combinations thereof.

13. The method of claim 1, wherein the at least one Trim28-specific inhibitory nucleic acid is administered daily for 6 weeks or more.

14. The method of claim 1, wherein the at least one Trim28-specific inhibitory nucleic acid is administered daily for 12 weeks or more.

15. A method for monitoring the therapeutic efficacy of a Trim28-specific inhibitory nucleic acid in a subject diagnosed with AML comprising:
(a) detecting H3K9 trimethylation levels in a test sample obtained from the subject after the subject has been administered the Trim28-specific inhibitory nucleic acid; and
(b) determining that the Trim28-specific inhibitory nucleic acid is effective when the H3K9 trimethylation levels in the test sample are reduced compared to that observed in a control sample obtained from the subject prior to administration of the Trim28-specific inhibitory nucleic acid.

16. The method of claim 15, wherein H3K9 trimethylation levels are detected via chromatin immunoprecipitation.

17. A method for inhibiting leukemic cell proliferation in a subject in need thereof, comprising administering to the subject a therapeutically effective amount of at least one Trim28-specific inhibitory nucleic acid, wherein the subject suffers from a disease or condition characterized by elevated expression levels and/or increased activity of TRIM28.

18. The method of claim 17, wherein the Trim28-specific inhibitory nucleic acid is complementary to a Trim28 protein domain selected from the group consisting of RBCC domain, HP1 protein binding domain (HP1BD), Plant Homeodomain (PHD) and Bromodomain.

19. The method of claim 17, wherein the Trim28-specific inhibitory nucleic acid is a shRNA or a sgRNA.

20. The method of claim 17, wherein the Trim28-specific inhibitory nucleic acid is a sgRNA or shRNA comprising a nucleic acid sequence selected from the group consisting of:
(SEQ ID NO: 1)
5′ TTACAGTAGACTGTTCGCTCTC 3′,
(SEQ ID NO: 2)
5′ TTCTGCACATCAGACACCTGGC 3′,
(SEQ ID NO: 3)
5′ TTGAACTGTTTGAACATGCGGC 3′,
(SEQ ID NO: 4)
5′ TTAACTTGTTGAATTGCTTGAA 3′,
(SEQ ID NO: 5)
5′ TTCAATAACAATAAGGTTGTAG 3′,
(SEQ ID NO: 6)
5′ TAGATCAACTTCTTAGAAAGCA 3′,
(SEQ ID NO: 7)
5′ CTACAGGCCGAGTGCAAACA 3′,
(SEQ ID NO: 8)
5′ GAGAGCGCCTGCGACCCGAG 3′,
(SEQ ID NO: 9)
5′ CCAGCGGGTGAAGTACACCA 3′,
and
(SEQ ID NO: 10)
5′ CCCAGCCACCAGCTACTGTG 3′.


SEQUENCE LISTING (23 sequences)

SEQ ID NO: 1 - DNA, 22 bp, Artificial Sequence
  Description of Artificial Sequence Synthetic oligonucleotide
ttacagtagactgttcgctctc

SEQ ID NO: 2 - DNA, 22 bp, Artificial Sequence
  Description of Artificial Sequence Synthetic oligonucleotide
ttctgcacatcagacacctggc

SEQ ID NO: 3 - DNA, 22 bp, Artificial Sequence
  Description of Artificial Sequence Synthetic oligonucleotide
ttgaactgtttgaacatgcggc

SEQ ID NO: 4 - DNA, 22 bp, Artificial Sequence
  Description of Artificial Sequence Synthetic oligonucleotide
ttaacttgttgaattgcttgaa

SEQ ID NO: 5 - DNA, 22 bp, Artificial Sequence
  Description of Artificial Sequence Synthetic oligonucleotide
ttcaataacaataaggttgtag

SEQ ID NO: 6 - DNA, 22 bp, Artificial Sequence
  Description of Artificial Sequence Synthetic oligonucleotide
tagatcaacttcttagaaagca

SEQ ID NO: 7 - DNA, 20 bp, Artificial Sequence
  Description of Artificial Sequence Synthetic oligonucleotide
ctacaggccgagtgcaaaca

SEQ ID NO: 8 - DNA, 20 bp, Artificial Sequence
  Description of Artificial Sequence Synthetic oligonucleotide
gagagcgcctgcgacccgag

SEQ ID NO: 9 - DNA, 20 bp, Artificial Sequence
  Description of Artificial Sequence Synthetic oligonucleotide
ccagcgggtgaagtacacca

SEQ ID NO: 10 - DNA, 20 bp, Artificial Sequence
  Description of Artificial Sequence Synthetic oligonucleotide
cccagccaccagctactgtg

SEQ ID NO: 11 - DNA, 3364 bp, Homo sapiens
agtgacgcagaggctggagacgactctacggcggcgaagagacgcgggttgaggaagagggacggattgcccatgcgcttgggcgcacagcggcccgcttctgtgtggtctggaggtggagctgagaggggaatcacactctataaaggttcgcataccccactggcggattcaattgcggcagtgacgtcacagaggccccgccccgcccccacaagagccccaccgacgtggggttggcggtggtggaaggactaggagttggcgcgtgcgtactggcggcctctcccgcaccgaccggcctgggccccgcccccgggcgtgaggcgcccaatgcgcgtgcgcggcggcgtcggcgccagttatttctgtcccgccccccggcctcggctctttctgcgagcgggcgcgcgggcgagcggttgtgcttgtgcttgtggcgcgtggtgcgggtttcggcggcggctgaggaagaagcgcgggcggcgccttcgggaggcgagcaggcagcagttggccgtgccgtagcagcgtcccgcgcgcggcgggcagcggcccaggaggcgcgtggcggcgctcggcctcgcggcggcggcggcggcagcggcccagcagttggcggcgagcgcgtctgcgcctgcgcggcgggccccgcgcccctcctccccccctgggcgcccccggcggcgtgtgaatggcggcctccgcggcggcagcctcggcagcagcggcctcggccgcctctggcagcccgggcccgggcgagggctccgctggcggcgaaaagcgctccaccgccccttcggccgcagcctcggcctctgcctcagccgcggcgtcgtcgcccgcggggggcggcgccgaggcgctggagctgctggagcactgcggcgtgtgcagagagcgcctgcgacccgagagggagccccgcctgctgccctgtttgcactcggcctgtagtgcctgcttagggcccgcggcccccgccgccgccaacagctcgggggacggcggggcggcgggcgacggcaccgtggtggactgtcccgtgtgcaagcaacagtgcttctccaaagacatcgtggagaattatttcatgcgtgatagtggcagcaaggctgccaccgacgcccaggatgcgaaccagtgctgcactagctgtgaggataatgccccagccaccagctactgtgtggagtgctcggagcctctgtgtgagacctgtgtagaggcgcaccagcgggtgaagtacaccaaggaccatactgtgcgctctactgggccagccaagtctcgggatggtgaacgtactgtctattgcaacgtacacaagcatgaaccccttgtgctgttttgtgagagctgtgatactctcacctgccgagactgccagctcaatgcccacaaggaccaccagtaccagttcttagaggatgcagtgaggaaccagcgcaagctcctggcctcactggtgaagcgccttggggacaaacatgcaacattgcagaagagcaccaaggaggttcgcagctcaatccgccaggtgtctgacgtacagaagcgtgtgcaagtggatgtcaagatggccatcctgcagatcatgaaggagctgaataagcggggccgtgtgctggtcaatgatgcccagaaggtgactgaggggcagcaggagcgcctggagcggcagcactggaccatgaccaagatccagaagcaccaggagcacattctgcgctttgcctcttgggctctggagagtgacaacaacacagcccttttgctttctaagaagttgatctacttccagctgcaccgggccctcaagatgattgtggatcccgtggagccacatggcgagatgaagtttcagtgggacctcaatgcctggaccaagagtgccgaggcctttggcaagattgtggcagagcgtcctggcactaactcaacaggccctgcacccatggcccctccaagagccccagggcccctgagcaagcagggctctggcagcagccagcccatggaggtgcaggaaggctatggctttgggtcaggagatgatccctactcaagtgcagagccccatgtgtcaggtgtgaaacggtcccgctcaggtgagggcgaggtgagcggccttatgcgcaaggtgccacgagtgagccttgaacgcctggacctggacctcacagctgacagccagccacccgtcttcaaggtcttcccaggcagtaccactgaggactacaaccttattgttattgaacgtggcgctgccgctgcagctaccggccagccagggactgcgcctgcaggaacccctggtgccccacccctggctggcatggccattgtcaaggaggaggagacggaggctgccattggagcccctcctactgccactgagggccctgagaccaaacctgtgcttatggctcttgcggagggtcctggtgctgagggtccccgcctggcctcacctagtggcagcaccagctcagggctggaggtggtggctcctgagggtacctcagccccaggtggtggcccgggaaccctggatgacagtgccaccatttgccgtgtctgccagaagccaggcgatctggttatgtgcaaccagtgtgagttttgtttccacctggactgtcacctgccggccctgcaggatgtaccaggggaggagtggagctgctcactctgccatgtgctccctgacctgaaggaggaggatggcagcctcagcctggatggtgcagacagcactggcgtggtggccaagctctcaccagccaaccagcggaaatgtgagcgtgtactgctggccctattctgtcacgaaccctgccgccccctgcatcagctggctaccgactccaccttctccctggaccagcccggtggcaccctggatctgaccctgatccgtgcccgcctccaggagaagttgtcacctccctacagctccccacaggagtttgcccaggatgtgggccgcatgttcaagcaattcaacaagttaactgaggacaaggcagacgtgcagtccatcatcggcctgcagcgcttcttcgagacgcgcatgaacgaggccttcggtgacaccaagttctctgctgtgctggtggagcccccgccgatgagcctgcctggtgctggcctgagttcccaggagctgtctggtggccctggtgatggcccctgaggctggagcccccatggccagcccagcctggctctgttctctgtcctgtcaccccatccccactcccctggtggcctgactcccactccctggtggccccatcccccagttcctcacgatatggtttttacttctgtggatttaataaaaacttcaccagtt

SEQ ID NO: 12 - protein, 835 aa, Homo sapiens
MAASAAAASAAAASAASGSPGPGEGSAGGEKRSTAPSAAASASASAAASSPAGGGAEALELLEHCGVCRERLRPEREPRLLPCLHSACSACLGPAAPAAANSSGDGGAAGDGTVVDCPVCKQQCFSKDIVENYFMRDSGSKAATDAQDANQCCTSCEDNAPATSYCVECSEPLCETCVEAHQRVKYTKDHTVRSTGPAKSRDGERTVYCNVHKHEPLVLFCESCDTLTCRDCQLNAHKDHQYQFLEDAVRNQRKLLASLVKRLGDKHATLQKSTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPAPMAPPRAPGPLSKQGSGSSQPMEVQEGYGFGSGDDPYSSAEPHVSGVKRSRSGEGEVSGLMRKVPRVSLERLDLDLTADSQPPVFKVFPGSTTEDYNLIVIERGAAAAATGQPGTAPAGTPGAPPLAGMAIVKEEETEAAIGAPPTATEGPETKPVLMALAEGPGAEGPRLASPSGSTSSGLEVVAPEGTSAPGGGPGTLDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSLDQPGGTLDLTLIRARLQEKLSPPYSSPQEFAQDVGRMFKQFNKLTEDKADVQSIIGLQRFFETRMNEAFGDTKFSAVLVEPPPMSLPGAGLSSQELSGGPGDGP

SEQ ID NO: 13 - DNA, 3255 bp, Mus musculus
ggacctgcgcgctcaagcacgcgggttgccagcctgggtgcaccgtgcggtgggcatgggagacgtcaagcacgcactctgcgggtgaattcctcaccagcagcattgctactctggatctagaggggcggcagcgagtggaagggagggccctggcgcgtgcgtaatctgcgccccgcccccaggctgcggtgcccaacgcgcgtgcgcggcgtcgtcggcgccagatatttctgtcccgcctccaggccgcggctctttctgcgagcggcgcgcggtcaggcggttgttcgcaagtcttgcggcgcgagtgtctcagaagcgagggaggaggcggcgctggtggcggcgccgtcgtctgtacagaggcccgcagccagcggcttggccgcatctcgacagcgcctggggcgcggcgggcgtcggcccaggagacgcgtggcggcgctcggcctcgcggcatcggcggctgcctggccgttggcggcgagcgcacttgcgcctgcgcagcgggctccgtgcccctcctcccctgggcggcccccccacccccccggcggcgtgtgaatggcggcctcggcggcagcgactgcagcggcctcggccgcgacggccgcctcggcggcctctggtagcccagggtcgggcgagggctcggcgggcggtgagaagcgtccggctgcttcctcagccgcggcggcctctgcagccgcgtcgtcccctgcggggggcggtggcgaggcgcaggagcttctggagcactgcggcgtgtgtcgcgagcgcctgcggcccgagcgggatcctcggctgctgccctgtctacattcggcctgcagtgcctgcctgggccccgctacacccgccgcagcgaataattcgggggatggcggctcggcgggcgacggcgctatggtggattgtccagtgtgcaaacagcagtgctactccaaagacatcgtggagaattattttatgcgtgatagtggcagtaaggcctcttctgattcccaggatgctaaccagtgctgcactagctgtgaagataatgccccagccactagctattgtgtggagtgctctgaaccactttgtgagacctgtgtggaggctcaccagcgggtgaaatacaccaaggaccacactgtgcgctccacaggacctgctaagactcgagatggagagcgaacagtctactgtaatgtgcacaagcatgagcccctcgtgctgttctgtgagagctgtgacacactcacctgccgcgactgccagctcaacgctcacaaggaccatcagtaccagtttttggaagatgcagtgaggaaccaacgtaaactcttggcttcactggtgaaacgtcttggggacaaacatgccacacttcagaaaaacaccaaggaggttcgaagctcgatccgccaggtgtctgatgtgcagaagcgagtgcaggttgatgtcaagatggccattctgcagatcatgaaggagctgaataagcggggtcgagttctggtcaatgatgcccagaaggtgaccgagggtcagcaggaacgtctggagcgccagcactggaccatgaccaaaattcagaagcaccaggaacacattttgcgttttgcctcttgggctctggagagtgataacaatacagctctcttgctctctaagaagctgatctatttccagctgcatcgggccctcaaaatgattgtggatcctgtggagcctcatggtgagatgaagtttcagtgggatctcaatgcctggaccaagagtgctgaagcctttggcaagattgtggctgagcgtcctggtacgaactccacaggtcctgggcccatggctcctccaagagccccaggccctctaagcaagcaaggttctggcagtagccagcccatggaagtacaagagggatatggctttgggtcagatgatccctattcaagtgcagagccgcatgtatcaggcatgaagcggtcccgctctggtgagggagaggtaagtggcctcttaaggaaggtgccacgtgtgagccttgaacgcctggatctggacctcacctctgacagccagccaccagtcttcaaggtctttcctggaagcactactgaggactacaatctgattgttattgagcgtggtgctgctgcagcagctgctggtcaggctgggactgtgccaccaggagcccctggtgccccaccccttcctggcatggccattgtcaaggaagaagagacagaagctgctattggagctcccccggctgcccccgagggtcctgaaaccaagcctgtgttgatgcctctgactgaaggtcctggtgccgagggacctcgtctagcttcacctagtggcagtaccagctcaggcttggaggtggtggctcctgaggttacctcagccccagtaagtgggccaggtatcctggatgacagtgccactatctgccgtgtctgccagaaaccaggtgacctggtcatgtgtaaccagtgcgaattttgcttccacctggattgccacctccctgccctgcaggatgttccaggggaggaatggagttgctcactctgccacgtgctccctgacctaaaggaggaagatggaagcctcagcctggatggagcagatagcactggtgtggtagctaaactctcaccagccaaccagcggaaatgtgagcgtgttctcctggccctgttctgccatgaaccatgccgtcccttgcatcagctggctaccgactctacattctccatggagcagcctggtggtaccctagacctgaccttgattcgtgctcgcctccaagagaagctgtcacctccttatagctccccccaggagtttgctcaagatgtgggccgcatgttcaaacagttcaacaagctgactgaggacaaggcagatgttcagtccatcatcggcttgcagcgcttctttgagacacgcatgaatgatgcctttggtgacaccaagttttctgctgtgctggtagaaccaccaccattgaaccttcccagtgctggcctaagttctcaggagctctctggccctggtgatggcccctgaagctggggctcttgtggtcagcccagtccagctctggtctctgtattttcaccccataccctgtcctttggtggcctgactcctgttcttgctggccccatcgtcccctcagtccctcttcacaaaatggtttttacttctgtggatttaataaaaacttcactgagtcaaaaaaaaaaaaaa

SEQ ID NO: 14 - protein, 834 aa, Mus musculus
MAASAAATAAASAATAASAASGSPGSGEGSAGGEKRPAASSAAAASAAASSPAGGGGEAQELLEHCGVCRERLRPERDPRLLPCLHSACSACLGPATPAAANNSGDGGSAGDGAMVDCPVCKQQCYSKDIVENYFMRDSGSKASSDSQDANQCCTSCEDNAPATSYCVECSEPLCETCVEAHQRVKYTKDHTVRSTGPAKTRDGERTVYCNVHKHEPLVLFCESCDTLTCRDCQLNAHKDHQYQFLEDAVRNQRKLLASLVKRLGDKHATLQKNTKEVRSSIRQVSDVQKRVQVDVKMAILQIMKELNKRGRVLVNDAQKVTEGQQERLERQHWTMTKIQKHQEHILRFASWALESDNNTALLLSKKLIYFQLHRALKMIVDPVEPHGEMKFQWDLNAWTKSAEAFGKIVAERPGTNSTGPGPMAPPRAPGPLSKQGSGSSQPMEVQEGYGFGSDDPYSSAEPHVSGMKRSRSGEGEVSGLLRKVPRVSLERLDLDLTSDSQPPVFKVFPGSTTEDYNLIVIERGAAAAAAGQAGTVPPGAPGAPPLPGMAIVKEEETEAAIGAPPAAPEGPETKPVLMPLTEGPGAEGPRLASPSGSTSSGLEVVAPEVTSAPVSGPGILDDSATICRVCQKPGDLVMCNQCEFCFHLDCHLPALQDVPGEEWSCSLCHVLPDLKEEDGSLSLDGADSTGVVAKLSPANQRKCERVLLALFCHEPCRPLHQLATDSTFSMEQPGGTLDLTLIRARLQEKLSPPYSSPQEFAQDVGRMFKQFNKLTEDKADVQSIIGLQRFFETRMNDAFGDTKFSAVLVEPPPLNLPSAGLSSQELSGPGDGP

SEQ ID NO: 15 - DNA, 137 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 16 - DNA, 123 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 17 - DNA, 110 bp, Artificial Sequence
  Description of Artificial Sequence Synthetic polynucleotide
  modified_base (1)..(20): a, c, t, g, unknown or other
nnnnnnnnnnnnnnnnnnnngtttttgtactctcagaaatgcagaagctacaaagataaggcttcatgccgaaatcaacaccctgtcattttatggcagggtgttttttt

SEQ ID NO: 18 - DNA, 102 bp, Artificial Sequence
  Description of Artificial Sequence Synthetic polynucleotide
  modified_base (1)..(20): a, c, t, g, unknown or other
nnnnnnnnnnnnnnnnnnnngttttagagctagaaatagcaagttaaaataaggctagtccgttatcaacttgaaaaagtggcaccgagtcggtgctttttt

SEQ ID NO: 19 - DNA, 88 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcaacttgaaaaagtgttttttt

SEQ ID NO: 20 - DNA, 76 bp, Artificial Sequence
  Description of Artificial Sequence Synthetic oligonucleotide
  modified_base (1)..(20): a, c, t, g, unknown or other
nnnnnnnnnnnnnnnnnnnngttttagagctagaaatagcaagttaaaataaggctagtccgttatcatttttttt

SEQ ID NO: 21 - DNA, 22 bp, Artificial Sequence
  Description of Artificial Sequence Synthetic oligonucleotide
ttattgacatcgttcaccttga

SEQ ID NO: 22 - DNA, 22 bp, Artificial Sequence
  Description of Artificial Sequence Synthetic oligonucleotide
tgtaacctcacattgaacctga

SEQ ID NO: 23 - DNA, 20 bp, Artificial Sequence
  Description of Artificial Sequence Synthetic oligonucleotide
gtacaaccctcctgaccatg